SEQUENCE LISTING (1 sequence)

SEQ ID NO: 1 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

(19) United States
(12) Patent Application Publication
(10) Pub. No.: US 2026/0048045 A1
(43) Pub. Date: Feb. 19, 2026

(54) PI3K-ALPHA INHIBITORS FOR THE TREATMENT OF CANCER

(71) Applicant: Scorpion Therapeutics, Inc., Boston, MA (US)

(72) Inventors: Natasja Brooijmans, Boston, MA (US); David St. Jean, Jr., Natick, MA (US)

(21) Appl. No.: 19/100,051

(22) PCT Filed: Jul. 31, 2023
(86) PCT No.: PCT/US2023/071333

§ 371 Date: Jan. 30, 2025

Related U.S. Application Data
(60) Provisional application No. 63/395,703, filed on Aug. 5, 2022.

(51) Int. Cl. A61K 31/443; A61K 31/4184; A61P 35/00; C07D 405/12; G01N 33/50; G16B 15/30; G16B 35/20

(52) U.S. Cl. CPC A61K 31/443; A61K 31/4184; A61P 35/00; C07D 405/12; G01N 33/5008; G16B 15/30; G16B 35/20; G01N 2500/04; G01N 2500/10

(57) ABSTRACT

This disclosure provides compounds and pharmaceutically acceptable salts thereof that inhibit phosphatidylinositol 4,5-bisphosphate 3-kinase (PI3K) isoform alpha (PI3Kα), as well as methods of screening for such compounds.

### CROSS REFERENCE TO RELATED APPLICATIONS

This application claims the benefit of U.S. Provisional Application No. 63/395,703, filed on Aug. 5, 2022, which is incorporated herein by reference in its entirety.

### SEQUENCE LISTING

This application contains a Sequence Listing that has been submitted electronically as an XML file named “50006-0088WO1_ST26_SL.XML.” The XML file, created on Jul. 31, 2023, is 2,934 bytes in size. The material in the XML file is hereby incorporated by reference in its entirety.

### TECHNICAL FIELD

This disclosure provides compounds and pharmaceutically acceptable salts thereof that inhibit phosphatidylinositol 4,5-bisphosphate 3-kinase (PI3K) isoform alpha (PI3Kα), as well as methods of screening for such compounds.

### BACKGROUND

Phosphorylation of target proteins by protein kinases is tightly regulated and any perturbation may lead to a disease state, such as cancer. As such, kinases are highly studied drug targets. Nearly all reported kinase inhibitors, including the currently approved kinase-targeting drugs for oncology, inhibit kinase activity via binding in the ATP binding site (i.e., orthosteric inhibitors). Zhang, et al., (2009) Nat. Rev. Cancer 9, 28-39. This approach leverages the ubiquitous ATP binding site, present in all kinases, which is a pocket well suited for binding small molecules.

However, the highly-conserved nature of the ATP binding pocket across the kinome leads to a primary drawback of targeting this binding site: lack of selectivity between different kinases. See, e.g., Davis, et al. (2011) Nat. Biotechnol. 29, 1046-U124. This lack of selectivity can result in toxicity due to off-target binding and may also require highly potent compounds in order to compete with millimolar intracellular ATP concentrations. Scapin (2006) Curr. Drug Targets 7, 1443-1454. Common mutations in and around the ATP pocket, including gatekeeper residues, can also confer resistance to inhibitors targeting this site. See Krishnamurty and Maly (2010) ACS Chem. Biol. 5, 121-138. Thus, there is a need for the development of kinase inhibitors that do not target the ATP binding site (e.g., allosteric inhibitors).

### SUMMARY

Some embodiments provide a PI3Kα inhibitor comprising a compound capable of forming a direct binding interaction with an allosteric pocket on PI3Kα revealed by displacing Phe937 and Leu938, thereby exposing the allosteric pocket;
wherein the allosteric pocket comprises Thr813, Leu911, and Phe1002;
wherein the compound forms direct binding interactions with one or more amino acids of the allosteric pocket; and
wherein the compound has an K_{D} for PI3Kα^{H1047R} of about 0.1 nM to about 1 μM.

Some embodiments provide a PI3Kα inhibitor comprising a compound that does form a direct binding interaction with an allosteric pocket on PI3Kα revealed by displacing Phe937 and Leu938, thereby exposing the allosteric pocket;
wherein the allosteric pocket comprises Thr813, Leu911, and Phe1002;
wherein the compound forms direct binding interactions with one or more amino acids of the allosteric pocket; and
wherein the compound has an K_{D} for PI3Kα^{H1047R} of about 0.1 nM to about 1 μM.

Some embodiments provide a PI3Kα inhibitor comprising a compound capable of forming a direct binding interaction with an allosteric pocket on PI3Kα;
wherein the allosteric pocket comprises Leu911, Phe937, and Phe1002;
wherein the compound forms direct binding interactions with one or more amino acids of the allosteric pocket; and
wherein the compound has an K_{D} for PI3Kα^{H1047R} of about 0.1 nM to about 1 μM.

Some embodiments provide a PI3Kα inhibitor comprising a compound that does form a direct binding interaction with an allosteric pocket on PI3Kα;
wherein the allosteric pocket comprises Leu911, Phe937, and Phe1002;
wherein the compound forms direct binding interactions with one or more amino acids of the allosteric pocket; and
wherein the compound has an K_{D} for PI3Kα^{H1047R} of about 0.1 nM to about 1 μM.

Some embodiments provide a PI3Kα inhibitor compound comprising:
(i) a first, a second, and a third hydrogen bonding moiety each capable of forming hydrogen bonds with Leu911 and/or Lys941 of PI3Kα^{H1047R}, and wherein the first and second, second and third, and first and third hydrogen bonding moieties are each about 2.4 Å apart;
(ii) a hydrophobic moiety about 2.5 Å from the third hydrogen bonding moiety and is capable of interacting with Ile1022 of PI3Kα^{H1047R}; and
(iii) a first and a second aromatic moiety, wherein the first and second aromatic moieties comprise a fused bicyclic aromatic ring system or two aromatic rings connected by a single bond, and wherein the first and second aromatic moieties are each capable of forming a pi-pi stacking interaction with Phe937 of PI3Kα^{H1047R}.
(iv) a third aromatic moiety capable of forming a cation-pi interaction with Lys941 and/or a pi-pi stacking interaction with Tyr1021 and/or a of PI3Kα^{H1047R}; and
(v) a fourth hydrogen bonding moiety capable of forming hydrogen bonds with Glu950 and/or Arg949;
wherein the PI3Kα inhibitor has a K_{D} for PI3Kα^{H1047R} of about 0.1 nM to about 1 μM.

Some embodiments provide a PI3Kα inhibitor compound comprising a first and a second aromatic moiety joined by a linking group to a third aromatic moiety, wherein the first and a second aromatic moieties comprise a fused bicyclic aromatic ring system or two aromatic rings connected by a single bond, wherein:
(i) the linking group is capable of forming hydrogen bonds with Leu911 and/or Lys941 of PI3Kα^{H1047R}.
(ii) the first and second aromatic moieties are each capable of forming a pi-pi stacking interaction with Phe937 of PI3Kα^{H1047R}; and
(iii) the third aromatic moiety is capable of forming an optional cation-pi interaction with Lys941 and/or a pi-pi stacking interaction with Tyr1021 of PI3Kα^{H1047R};
wherein the PI3Kα inhibitor has a K_{D} for PI3Kα^{H1047R} of about 0.1 nM to about 1 μM.

Also provided herein is a method of identifying a PI3Kα inhibitor compound comprising:
(i) screening in silico a library for candidate compounds capable of forming a direct binding interaction with an allosteric pocket on PI3Kα, wherein a three-dimensional model of the binding site on PI3Kα is computationally derived from the atomic coordinates in Table 1; and
(ii) evaluating the candidate compounds identified in step (i) in one or more in vitro or in vivo assays for their ability to bind to the PI3Kα allosteric pocket to thereby identify the PI3Kα inhibitor;
wherein the PI3Kα inhibitor compound has a K_{D} for PI3Kα^{H1047R} of about 0.1 nM to about 1 μM.

Also provided herein is a method of identifying a PI3Kα inhibitor compound comprising:
(i) using the atomic coordinates in Table 2 to generate a 3-dimensional model of PI3Kα^{H1047R}.
(ii) identifying three or more residues of an allosteric binding pocket;
(ii) generating a specific 3-dimensional target using the three or more allosteric binding pocket residues;
(iii) employing the specific 3-dimensional target to select a potential inhibitor of PI3Kα;
(iv) obtaining the potential inhibitor of PI3Kα; and
(v) contacting the potential inhibitor of PI3Kα with PI3Kα^{H1047R} in vitro to determine a K_{D} for PI3Kα^{H1047R}, wherein if the K_{D} for PI3Kα^{H1047R} is about 0.1 nM to about 1 μM, the potential inhibitor of PI3Kα is a PI3Kα inhibitor.

Also provided herein is a pharmaceutical composition comprising a compound of Formula (I), or a pharmaceutically acceptable salt thereof, and a pharmaceutically acceptable carrier.

Provided herein is a method for treating cancer in a subject in need thereof, comprising administering to the subject a therapeutically effective amount of a compound of Formula (I), or a pharmaceutically acceptable salt thereof, or a pharmaceutical composition as provided herein.

This disclosure also provides a method for inhibiting PI3Kα in a mammalian cell, the method comprising contacting the mammalian cell with an effective amount of a compound of Formula (II), or a pharmaceutically acceptable salt thereof.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The term “about” when referring to a number or a numerical range means that the number or numerical range referred to is an approximation, for example, within experimental variability and/or statistical experimental error, and thus the number or numerical range may vary up to ±10% of the stated number or numerical range.

The term “acceptable” with respect to a formulation, composition or ingredient, as used herein, means having no persistent detrimental effect on the general health of the subject being treated.

The term “inhibit” or “inhibition of” means to reduce by a measurable amount, or to prevent entirely (e.g., 100% inhibition).

“API” refers to an active pharmaceutical ingredient.

The term “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, a “therapeutically effective amount” means an amount of compound that, when administered to a subject in need of such treatment, is sufficient to (i) treat a PI3Kα protein-associated disease or disorder, (ii) attenuate, ameliorate, or eliminate one or more symptoms of the particular disease, condition, or disorder, or (iii) delay the onset of one or more symptoms of the particular disease, condition, or disorder described herein. An appropriate “therapeutically effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt is not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The term “eudysmic ratio” refers to the difference in activity (e.g., IC₅₀, EC₅₀, K_{D}, and the like) between two enantiomers of a compound.

The term “cation-pi interaction” refers to a stabilizing electrostatic interaction of a fully or partially positively charged group (i.e., cation) with the electron-rich, polarizable pi electrons of an aromatic moiety, as defined herein. Exemplary cations include, but are not limited to, tertiary amines and protonated amino groups such as those in the side chains of lysine and arginine residues. Exemplary amino acids containing an aromatic moiety include, but are not limited to, phenylalanine, tyrosine, and tryptophan.

The term “hydrogen bonding moiety” refers to groups capable of forming hydrogen bonds, such as hydrogen bond donors, and hydrogen bond acceptors. Exemplary hydrogen bonding moieties include, but are not limited to hydroxyl groups, ethers, amino groups (e.g., primary and secondary amines), perfluoro alkyl groups, carboxylic acids, oxo groups (including aldehydes, ketones, amides, carbamates, ureas, and the like), sulfoxides, sulfonamides, and the like.

The term “hydrophobic moiety” refers to a non-polar group such as alkyl groups, cycloalkyl groups, aryl groups, and the like.

The term “halo” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “backbone carbonyl” refers to a carbonyl group that participates in forming the amide bonds between amino acid residues in a peptide or protein (i.e., not including side chain carbonyl groups such as in the side chain of glutamine).

The term “hydroxyl” refers to an —OH radical.

The term “amide” refers to a —NH—C(═O)— or a —C(═O)NH— group.

The term “carbamate” refers to a —NHC(═O)O— or a —OC(═O)NH— group.

The term “urea” refers to a —NHC(═O)NH— group.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo.

The term “cycloalkyl” as used herein refers to cyclic saturated hydrocarbon groups having, e.g., 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like. The term “saturated” as used in this context means only single bonds present between constituent carbon atoms.

The term “aromatic moiety” refers to aryl and heteroaryl groups, as defined herein.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “heteroaryl”, as used herein, means a mono-, bi-, tri- or polycyclic group having 5 to 20 ring atoms, alternatively 5, 6, 9, 10, or 14 ring atoms; wherein at least one ring in the system contains one or more heteroatoms independently selected from the group consisting of N, O, and S and at least one ring in the system is aromatic (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

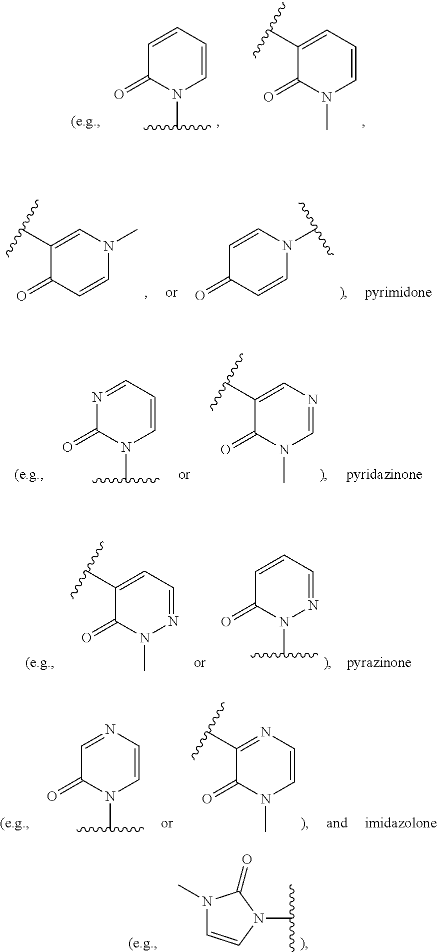

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic saturated or partially unsaturated ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein one or more ring atoms may be substituted by 1-3 oxo (forming, e.g., a lactam) and one or more N or S atoms may be substituted by 1-2 oxido (forming, e.g., an N-oxide, an S-oxide, or an S,S-dioxide), valence permitting; and wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, tetrahydropyridyl, dihydropyrazinyl, dihydropyridyl, dihydropyrrolyl, dihydrofuranyl, dihydrothiophenyl, and the like. Heterocyclyl may include multiple fused and bridged rings. Non-limiting examples of fused/bridged heterocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, examples of aromatic moieties include, but are not limited to: benzene, pyridine, pyrimidine, pyrazine, pyridazine, pyridone, pyrrole, pyrazole, oxazole, thioazole, isoxazole, isothiazole, imidazole, and their fused, bicyclic combinations.

As used herein, when a ring is described as being “partially unsaturated”, it means said ring has one or more additional degrees of unsaturation (in addition to the degree of unsaturation attributed to the ring itself; e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

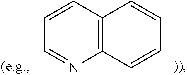

(ii) a single ring atom (spiro-fused ring systems)

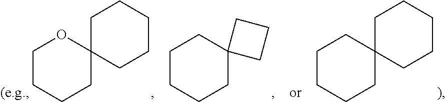

or (iii) a contiguous array of ring atoms (bridged ring systems having all bridge lengths>0)

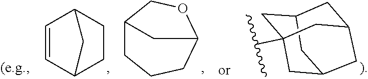

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

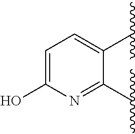 [SMILES: *c1ccc(O)nc1*]

encompasses the tautomeric form containing the moiety:

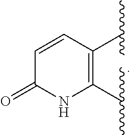 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass enantiomers (e.g., R and S isomers), diastereomers, as well as mixtures of enantiomers (e.g., R and S isomers) including racemic mixtures and mixtures of diastereomers, as well as individual enantiomers and diastereomers, which arise as a consequence of structural asymmetry in certain compounds. Unless otherwise indicated, when a disclosed compound is named or depicted by a structure without specifying the stereochemistry (e.g., a “flat” structure) and has one or more chiral centers, it is understood to represent all possible stereoisomers of the compound.

The details of one or more embodiments of this disclosure are set forth in the accompanying drawings and the description below. Other features and advantages of the present disclosure will be apparent from the description and drawings, and from the claims.

### BRIEF DESCRIPTION OF THE FIGURES

FIG. 1 depicts the surface of a PI3Kα inhibitor described herein bound to the allosteric pocket of PI3Kα. Relevant portions of the activation loop are also shown, along with the 1047-containing helix, i.e., helix kα11.

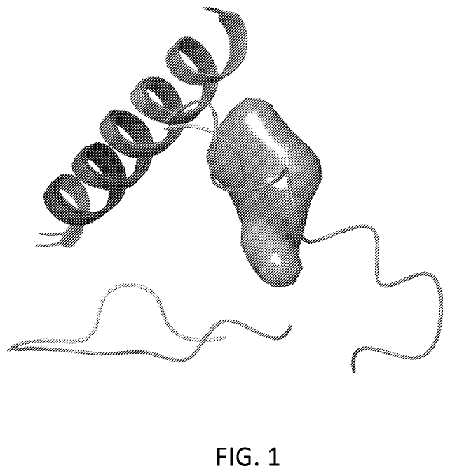

FIG. 2 illustrates the movement of the activation loop and the 1047-containing helix from the native confirmation (light gray) when the activation loop falls across the allosteric pocket, precluding binding to that site, to the conformation when a PI3Kα inhibitor described herein is bound to the allosteric site, stabilizing the activation loop in the “open” conformation.

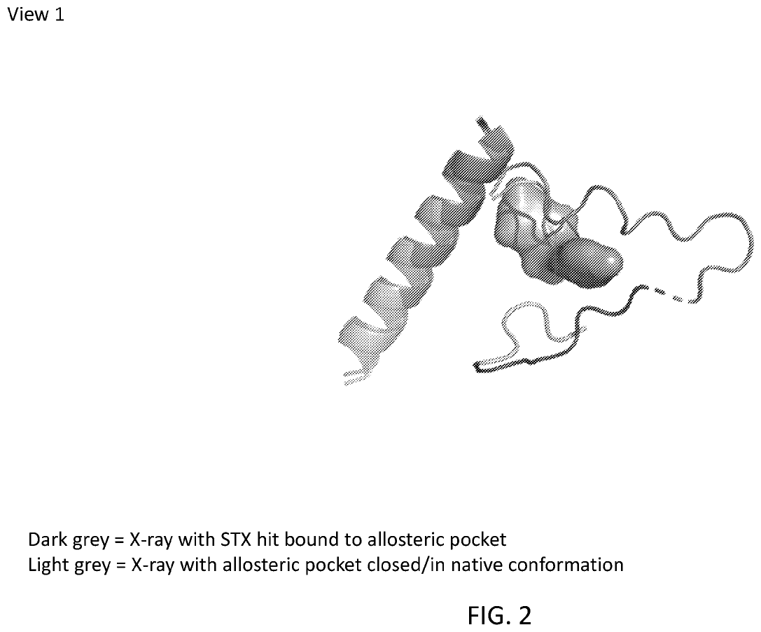

FIG. 3 provides the same view of the compound, activation loop, and 1047-containing helix as in FIG. 2, with the specific manipulations made to arrive at FIG. 4 (90 degree rotation around the X axis) and FIG. 5 (90 degree rotation around the Y axis).

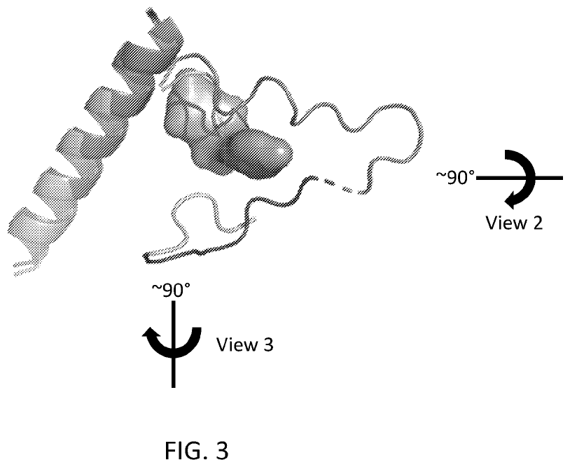

FIG. 4 illustrates the movement of the activation loop in a “top down” perspective from the unbound (closed) conformation to the bound (open) conformation.

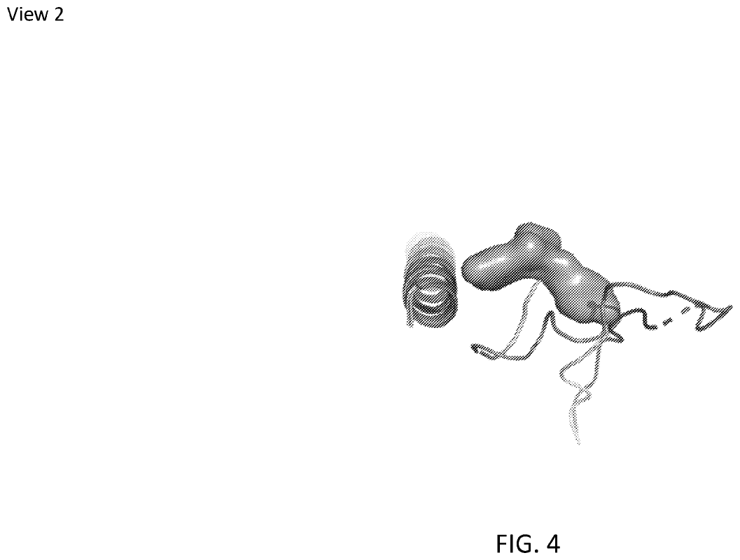

FIG. 5 illustrates a side view of the movement of the activation loop from the unbound (closed) conformation to the bound (open) conformation.

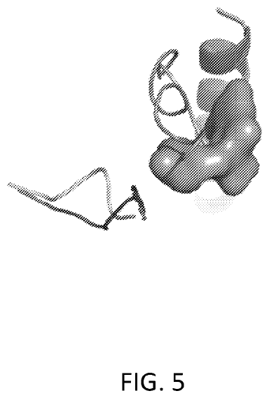

### DETAILED DESCRIPTION

Most allosteric kinase inhibitors to-date were discovered by accident, not by design. The present disclosure describes compounds that bind to a novel allosteric pocket on the phosphatidylinositol 4,5-bisphosphate 3-kinase (PI3K) isoform alpha (PI3Kα), e.g., PI3Kα^{H1047R}, to inhibit PI3K activity. Without being bound by any theory, movement of the activation loop in PI3K reveals this allosteric site, which when bound with a compound described herein, stabilizes the activation loop in a catalytically incompetent conformation resulting in inhibition of enzymatic activity.

Phosphatidylinositol 4,5-bisphosphate 3-kinase (PI3K)

PI3K is encoded by the PIK3CA gene and is part of the PI3K/AKT/TOR signaling network is altered in several human cancers. PI3K/AKT signaling is involved in physiological and pathophysiological functions that drive tumor progression such as metabolism, cell growth, proliferation, angiogenesis and metastasis. (See, Fruman, D. A. The PI3K Pathway in Human Disease. Cell 2017, 170, 605-635 and Janku, F. et al., Targeting the PI3K pathway in cancer: Are we making headway? Nat. Rev. Clin. Oncol. 2018, 15, 273-291.) Suppression (e.g., pharmacological or genetic) of PI3K/AKT/TOR signaling may cause cancer cell death and regression of tumor growth.

Exemplary Sequence of Human Phosphatidylinositol 4,5-bisphosphate 3-kinase isoform alpha (UniProtKB entry P42336) (SEQ ID NO: 1)

MPPRPSSGEL WGIHLMPPRI LVECLLPNGM IVTLECLREA
TLITIKHELF KEARKYPLHQ LLQDESSYIF VSVTQEAERE
EFFDETRRLC DLRLFQPFLK VIEPVGNREE KILNREIGFA
IGMPVCEFDM VKDPEVQDFR RNILNVCKEA VDLRDLNSPH
SRAMYVYPPN VESSPELPKH IYNKLDKGQI IVVIWVIVSP
NNDKQKYTLK INHDCVPEQV IAEAIRKKTR SMLLSSEQLK
LCVLEYQGKY ILKVCGCDEY FLEKYPLSQY KYIRSCIMLG
RMPNLMLMAK ESLYSQLPMD CFTMPSYSRR ISTATPYMNG
ETSTKSLWVI NSALRIKILC ATYVNVNIRD IDKIYVRTGI
YHGGEPLCDN VNTQRVPCSN PRWNEWLNYD IYIPDLPRAA
RLCLSICSVK GRKGAKEEHC PLAWGNINLF DYTDTLVSGK
MALNLWPVPH GLEDLLNPIG VTGSNPNKET PCLELEFDWF
SSVVKFPDMS VIEEHANWSV SREAGFSYSH AGLSNRLARD
NELRENDKEQ LKAISTRDPL SEITEQEKDF LWSHRHYCVT
IPEILPKLLL SVKWNSRDEV AQMYCLVKDW PPIKPEQAME
LLDCNYPDPM VRGFAVRCLE KYLTDDKLSQ YLIQLVQVLK
YEQYLDNLLV RFLLKKALTN QRIGHFFFWH LKSEMHNKTV
SQRFGLLLES YCRACGMYLK HLNRQVEAME KLINLTDILK
QEKKDETQKV QMKFLVEQMR RPDFMDALQG FLSPLNPAHQ
LGNLRLEECR IMSSAKRPLW LNWENPDIMS ELLFQNNEII
FKNGDDLRQD MLTLQIIRIM ENIWQNQGLD LRMLPYGCLS
IGDCVGLIEV VRNSHTIMQI QCKGGLKGAL QFNSHTLHQW
LKDKNKGEIY DAAIDLFTRS CAGYCVATFI LGIGDRHNSN
IMVKDDGQLF HIDFGHFLDH KKKKFGYKRE RVPFVLTQDF
LIVISKGAQE CTKTREFERF QEMCYKAYLA IRQHANLFIN
LFSMMLGSGM PELQSFDDIA YIRKTLALDK TEQEALEYFM
KQMNDAHHGG WTTKMDWIFH TIKQHALN

The PI3K pathway can be activated via, for example, point mutation(s) of the PIK3CA gene or via inactivation of the phosphatase and tensin homolog (PTEN) gene. Activation of this pathway occurs in approximately 30-50% human cancers and contributes to resistance to various anti-cancer therapies. (See, Martini, M. et al., PI3K/AKT signaling pathway and cancer: An updated review. Ann. Med. 2014, 46, 372-383 and Bauer, T. M. et al., Targeting PI3 kinase in cancer. Pharmacol. Ther. 2015, 146, 53-60.) PI3K consists of three subunits: p85 regulatory subunit, p55 regulatory subunit, and p110 catalytic subunit. According to their different structures and specific substrates, PI3K is divided into 3 classes: classes I, II, and III. Class I PI3Ks include class IA and class IB PI3Ks. Class IA PI3K, a heterodimer of p85 regulatory subunit and p110 catalytic subunit, is the type most clearly implicated in human cancer. Class IA PI3K includes p110a, p1100 and p1106 catalytic subunits produced from different genes (PIK3CA, PIK3CB and PIK3CD, respectively), while p110γ produced by PIK3CG represents the only catalytic subunit in class IB PI3K. PIK3CA, the gene encoding the p110α subunit, is frequently mutated or amplified in many human cancers, such as breast cancer, colon cancer, gastric cancer, cervical cancer, prostate cancer, and lung cancer. (See, Samuels Y, et al. High frequency of mutations of the PIK3CA gene in human cancers. Science. 2004; 304:554.)

However, the development of PI3K inhibitors has been problematic for several reasons including (i) adaptive molecular mechanisms upon therapeutic inhibition of PI3K, (ii) inability to specifically inhibit signaling by PIK3CA mutations while sparing endogenous p110α, (iii) the limited use of these therapies in rational combinations, including those informed with strong mechanistic support, and (iv) dose-limiting toxicities that prevent sustained PI3K pathway suppression. (See, Hanker et al., Challenges for the Clinical Development of PI3K Inhibitors: strategies to Improve Their Impact in solid Tumors, Cancer Discovery, April 2019; 9: 482-491.) Additionally, there are other factors and compensatory pathways derived from both clinical and in vitro lab studies, which affect PI3K signaling, such as HRAS and KRAS mutations, which reduce susceptibility to PI3K inhibitors (and knockdown of these has shown to improve sensitivity to PI3K inhibitors). (See, Misrha, R.; PI3K Inhibitors in Cancer: Clinical Implications and Adverse Effects. Int. J. Mol. Sci. 2021, 22, 3464.) Thus, novel PI3Kα inhibitors represent an approach for the treatment of proliferative disorders such as cancer.

### Compounds

Some embodiments provide a PI3Kα inhibitor comprising a compound capable of forming a direct binding interaction with an allosteric pocket on PI3Kα revealed by displacing Phe937 and Leu938, thereby exposing the allosteric pocket;
wherein the allosteric pocket comprises Thr813, Leu911, and Phe1002;
wherein the compound forms direct binding interactions with one or more amino acids of the allosteric pocket; and
wherein the compound has an K_{D} for PI3Kα^{H1047R} of about 0.1 nM to about 1 μM.

Some embodiments provide a PI3Kα inhibitor comprising a compound that does form a direct binding interaction with an allosteric pocket on PI3Kα revealed by displacing Phe937 and Leu938, thereby exposing the allosteric pocket;
wherein the allosteric pocket comprises Thr813, Leu911, and Phe1002;
wherein the compound forms direct binding interactions with one or more amino acids of the allosteric pocket; and
wherein the compound has an K_{D} for PI3Kα^{H1047R} of about 0.1 nM to about 1 μM.

In some embodiments, the allosteric pocket further comprises Phe937.

Some embodiments provide a PI3Kα inhibitor comprising a compound capable of forming a direct binding interaction with an allosteric pocket on PI3Kα;
wherein the allosteric pocket comprises Leu911, Phe937, and Phe1002;
wherein the compound forms direct binding interactions with one or more amino acids of the allosteric pocket; and
wherein the compound has an K_{D} for PI3Kα^{H1047R} of about 0.1 nM to about 1 μM.

Some embodiments provide a PI3Kα inhibitor comprising a compound that does form a direct binding interaction with an allosteric pocket on PI3Kα;
wherein the allosteric pocket comprises Leu911, Phe937, and Phe1002;
wherein the compound forms direct binding interactions with one or more amino acids of the allosteric pocket; and
wherein the compound has a K_{D} for PI3Kα^{H1047R} of about 0.1 nM to about 1 μM.

In some embodiments, the allosteric pocket further comprises Thr813.

In some embodiments, the allosteric pocket further comprises Lys941.

In some embodiments, the allosteric pocket further comprises one to five amino acids selected from: Arg949, Glu950, Val952, Tyr1021, and Ile1022.

In some embodiments, the allosteric pocket further comprises one of Arg949, Glu950, Val952, Tyr1021, and Ile1022. In some embodiments, the allosteric pocket further comprises Arg949. In some embodiments, the allosteric pocket further comprises Glu950. In some embodiments, the allosteric pocket further comprises Val952. In some embodiments, the allosteric pocket further comprises Tyr1021. In some embodiments, the allosteric pocket further comprises Ile1022.

In some embodiments, the allosteric pocket further comprises two of Arg949, Glu950, Val952, Tyr1021, and Ile1022. In some embodiments, the allosteric pocket further comprises Arg949 and Glu950. In some embodiments, the allosteric pocket further comprises Arg949 and Val952. In some embodiments, the allosteric pocket further comprises Arg949 and Tyr1021. In some embodiments, the allosteric pocket further comprises Arg949 and Ile1022. In some embodiments, the allosteric pocket further comprises Glu950 and Val952. In some embodiments, the allosteric pocket further comprises Glu950 and Tyr1021. In some embodiments, the allosteric pocket further comprises Glu950 and Ile1022. In some embodiments, the allosteric pocket further comprises Val952 and Tyr1021. In some embodiments, the allosteric pocket further comprises Val952 and Ile1022. In some embodiments, the allosteric pocket further comprises Tyr1021 and Ile1022.

In some embodiments, the allosteric pocket further comprises three of Arg949, Glu950, Val952, Tyr1021, and Ile1022. In some embodiments, the allosteric pocket further comprises Arg949, Glu950, and Val952. In some embodiments, the allosteric pocket further comprises Glu950, Val952, and Tyr1021. In some embodiments, the allosteric pocket further comprises Val952, Tyr1021, and Ile1022. In some embodiments, the allosteric pocket further comprises Arg949, Val952, and Tyr1021. In some embodiments, the allosteric pocket further comprises Arg949, Val952, and Ile1022. In some embodiments, the allosteric pocket further comprises Arg949, Tyr1021, and Ile1022. In some embodiments, the allosteric pocket further comprises Glu950, Tyr1021, and Ile1022. In some embodiments, the allosteric pocket further comprises Val952, Ile1022, and Glu950. In some embodiments, the allosteric pocket further comprises Glu950, Tyr1021, and Arg949. In some embodiments, the allosteric pocket further comprises Glu950, Ile1022, and Arg949.

In some embodiments, the allosteric pocket further comprises four of Arg949, Glu950, Val952, Tyr1021, and Ile1022. In some embodiments, the allosteric pocket further comprises Arg949, Glu950, Val952, and Tyr1021. In some embodiments, the allosteric pocket further comprises Glu950, Val952, Tyr1021, and Ile1022. In some embodiments, the allosteric pocket further comprises Val952, Tyr1021, Ile1022, and Arg949. In some embodiments, the allosteric pocket further comprises Tyr1021, Ile1022, Arg949, and Glu950. In some embodiments, the allosteric pocket further comprises Arg949, Glu950, Val952, and Ile1022.

In some embodiments, the allosteric pocket further comprises one or more of Gln809, Leu812, Ile816, Gly912, Leu938, His940, Arg951, Met1010, Glu1012, Leu1013, Asp1018, and Ile1019.

In some embodiments, the allosteric pocket further comprises one of Gln809, Leu812, Ile816, Gly912, Leu938, His940, Arg951, Met1010, Glu1012, Leu1013, Asp1018, and Ile1019. In some embodiments, the allosteric pocket further comprises Gln809. In some embodiments, the allosteric pocket further comprises Leu812. In some embodiments, the allosteric pocket further comprises Ile816. In some embodiments, the allosteric pocket further comprises Gly912. In some embodiments, the allosteric pocket further comprises Leu938. In some embodiments, the allosteric pocket further comprises His940. In some embodiments, the allosteric pocket further comprises Arg951. In some embodiments, the allosteric pocket further comprises Met1010. In some embodiments, the allosteric pocket further comprises Glu1012. In some embodiments, the allosteric pocket further comprises Leu1013. In some embodiments, the allosteric pocket further comprises Asp1018. In some embodiments, the allosteric pocket further comprises Ile1019. In some embodiments, the allosteric pocket further comprises two of Gln809, Leu812, Ile816, Gly912, Leu938, His940, Arg951, Met1010, Glu1012, Leu1013, Asp1018, and Ile1019.

In some embodiments, the allosteric pocket further comprises three of Gln809, Leu812, Ile816, Gly912, Leu938, His940, Arg951, Met1010, Glu1012, Leu1013, Asp1018, and Ile1019. In some embodiments, the allosteric pocket further comprises four of Gln809, Leu812, Ile816, Gly912, Leu938, His940, Arg951, Met1010, Glu1012, Leu1013, Asp1018, and Ile1019. In some embodiments, the allosteric pocket further comprises five of Gln809, Leu812, Ile816, Gly912, Leu938, His940, Arg951, Met1010, Glu1012, Leu1013, Asp1018, and Ile1019. In some embodiments, the allosteric pocket further comprises six of Gln809, Leu812, Ile816, Gly912, Leu938, His940, Arg951, Met1010, Glu1012, Leu1013, Asp1018, and Ile1019. In some embodiments, the allosteric pocket further comprises seven of Gln809, Leu812, Ile816, Gly912, Leu938, His940, Arg951, Met1010, Glu1012, Leu1013, Asp1018, and Ile1019. In some embodiments, the allosteric pocket further comprises eight of Gln809, Leu812, Ile816, Gly912, Leu938, His940, Arg951, Met1010, Glu1012, Leu1013, Asp1018, and Ile1019. In some embodiments, the allosteric pocket further comprises nine of Gln809, Leu812, Ile816, Gly912, Leu938, His940, Arg951, Met1010, Glu1012, Leu1013, Asp1018, and Ile1019. In some embodiments, the allosteric pocket further comprises ten of Gln809, Leu812, Ile816, Gly912, Leu938, His940, Arg951, Met1010, Glu1012, Leu1013, Asp1018, and Ile1019. In some embodiments, the allosteric pocket further comprises eleven of Gln809, Leu812, Ile816, Gly912, Leu938, His940, Arg951, Met1010, Glu1012, Leu1013, Asp1018, and Ile1019. In some embodiments, the allosteric pocket further comprises Gln809, Leu812, Ile816, Gly912, Leu938, His940, Arg951, Met1010, Glu1012, Leu1013, Asp1018, and Ile1019.

In some embodiments, the compounds described herein form a direct binding interaction with Thr813.

In some embodiments, the compounds described herein form a direct binding interaction with Lys941.

In some embodiments, the compounds described herein form a direct binding interaction with one to five amino acids selected from: Arg949, Glu950, Val952, Tyr1021, and Ile1022.

In some embodiments, the compounds described herein form a direct binding interaction with one of Arg949, Glu950, Val952, Tyr1021, and Ile1022. In some embodiments, the compounds described herein form a direct binding interaction with Arg949. In some embodiments, the compounds described herein form a direct binding interaction with Glu950. In some embodiments, the compounds described herein form a direct binding interaction with Val952. In some embodiments, the compounds described herein form a direct binding interaction with Tyr1021. In some embodiments, the compounds described herein form a direct binding interaction with Ile1022.

In some embodiments, the compounds described herein form a direct binding interaction with two of Arg949, Glu950, Val952, Tyr1021, and Ile1022. In some embodiments, the compounds described herein form a direct binding interaction with Arg949 and Glu950. In some embodiments, the compounds described herein form a direct binding interaction with Arg949 and Val952. In some embodiments, the compounds described herein form a direct binding interaction with Arg949 and Tyr1021. In some embodiments, the compounds described herein form a direct binding interaction with Arg949 and Ile1022. In some embodiments, the compounds described herein form a direct binding interaction with Glu950 and Val952. In some embodiments, the compounds described herein form a direct binding interaction with Glu950 and Tyr1021. In some embodiments, the compounds described herein form a direct binding interaction with Glu950 and Ile1022. In some embodiments, the compounds described herein form a direct binding interaction with Val952 and Tyr1021. In some embodiments, the compounds described herein form a direct binding interaction with Val952 and Ile1022. In some embodiments, the compounds described herein form a direct binding interaction with Tyr1021 and Ile1022.

In some embodiments, the compounds described herein form a direct binding interaction with three of Arg949, Glu950, Val952, Tyr1021, and Ile1022. In some embodiments, the compounds described herein form a direct binding interaction with Arg949, Glu950, and Val952. In some embodiments, the compounds described herein form a direct binding interaction with Glu950, Val952, and Tyr1021. In some embodiments, the compounds described herein form a direct binding interaction with Val952, Tyr1021, and Ile1022. In some embodiments, the compounds described herein form a direct binding interaction with Arg949, Val952, and Tyr1021. In some embodiments, the compounds described herein form a direct binding interaction with Arg949, Val952, and Ile1022. In some embodiments, the compounds described herein form a direct binding interaction with Arg949, Tyr1021, and Ile1022. In some embodiments, the compounds described herein form a direct binding interaction with Glu950, Tyr1021, and Ile1022. In some embodiments, the compounds described herein form a direct binding interaction with Val952, Ile1022, and Glu950. In some embodiments, the compounds described herein form a direct binding interaction with Glu950, Tyr1021, and Arg949. In some embodiments, the compounds described herein form a direct binding interaction with Glu950, Ile1022, and Arg949.

In some embodiments, the compounds described herein form a direct binding interaction with four of Arg949, Glu950, Val952, Tyr1021, and Ile1022. In some embodiments, the compounds described herein form a direct binding interaction with Arg949, Glu950, Val952, and Tyr1021. In some embodiments, the compounds described herein form a direct binding interaction with Glu950, Val952, Tyr1021, and Ile1022. In some embodiments, the compounds described herein form a direct binding interaction with Val952, Tyr1021, Ile1022, and Arg949. In some embodiments, the compounds described herein form a direct binding interaction with Tyr1021, Ile1022, Arg949, and Glu950. In some embodiments, the compounds described herein form a direct binding interaction with Arg949, Glu950, Val952, and Ile1022.

In some embodiments, the compounds described herein form a direct binding interaction with one or more of Gln809, Leu812, Ile816, Gly912, Leu938, His940, Arg951, Met1010, Glu1012, Leu1013, Asp1018, and Ile1019.

In some embodiments, the compounds described herein form a direct binding interaction with one of Gln809, Leu812, Ile816, Gly912, Leu938, His940, Arg951, Met1010, Glu1012, Leu1013, Asp1018, and Ile1019. In some embodiments, the compounds described herein form a direct binding interaction with Gln809. In some embodiments, the compounds described herein form a direct binding interaction with Leu812. In some embodiments, the compounds described herein form a direct binding interaction with Ile816. In some embodiments, the compounds described herein form a direct binding interaction with Gly912. In some embodiments, the compounds described herein form a direct binding interaction with Leu938. In some embodiments, the compounds described herein form a direct binding interaction with His940. In some embodiments, the compounds described herein form a direct binding interaction with Arg951. In some embodiments, the compounds described herein form a direct binding interaction with Met1010. In some embodiments, the compounds described herein form a direct binding interaction with Glu1012. In some embodiments, the compounds described herein form a direct binding interaction with Leu1013. In some embodiments, the compounds described herein form a direct binding interaction with Asp1018. In some embodiments, the compounds described herein form a direct binding interaction with Ile1019. In some embodiments, the compounds described herein form a direct binding interaction with two of Gln809, Leu812, Ile816, Gly912, Leu938, His940, Arg951, Met1010, Glu1012, Leu1013, Asp1018, and Ile1019.

In some embodiments, the compounds described herein form a direct binding interaction with three of Gln809, Leu812, Ile816, Gly912, Leu938, His940, Arg951, Met1010, Glu1012, Leu1013, Asp1018, and Ile1019. In some embodiments, the compounds described herein form a direct binding interaction with four of Gln809, Leu812, Ile816, Gly912, Leu938, His940, Arg951, Met1010, Glu1012, Leu1013, Asp1018, and Ile1019. In some embodiments, the compounds described herein form a direct binding interaction with five of Gln809, Leu812, Ile816, Gly912, Leu938, His940, Arg951, Met1010, Glu1012, Leu1013, Asp1018, and Ile1019. In some embodiments, the compounds described herein form a direct binding interaction with six of Gln809, Leu812, Ile816, Gly912, Leu938, His940, Arg951, Met1010, Glu1012, Leu1013, Asp1018, and Ile1019. In some embodiments, the compounds described herein form a direct binding interaction with seven of Gln809, Leu812, Ile816, Gly912, Leu938, His940, Arg951, Met1010, Glu1012, Leu1013, Asp1018, and Ile1019. In some embodiments, the compounds described herein form a direct binding interaction with eight of Gln809, Leu812, Ile816, Gly912, Leu938, His940, Arg951, Met1010, Glu1012, Leu1013, Asp1018, and Ile1019. In some embodiments, the compounds described herein form a direct binding interaction with nine of Gln809, Leu812, Ile816, Gly912, Leu938, His940, Arg951, Met1010, Glu1012, Leu1013, Asp1018, and Ile1019. In some embodiments, the compounds described herein form a direct binding interaction with ten of Gln809, Leu812, Ile816, Gly912, Leu938, His940, Arg951, Met1010, Glu1012, Leu1013, Asp1018, and Ile1019. In some embodiments, the compounds described herein form a direct binding interaction with eleven of Gln809, Leu812, Ile816, Gly912, Leu938, His940, Arg951, Met1010, Glu1012, Leu1013, Asp1018, and Ile1019. In some embodiments, the compounds described herein form a direct binding interaction with Gln809, Leu812, Ile816, Gly912, Leu938, His940, Arg951, Met1010, Glu1012, Leu1013, Asp1018, and Ile1019.

Some embodiments provide a PI3Kα inhibitor compound comprising:
(i) a first, a second, and a third hydrogen bonding moiety each capable of forming hydrogen bonds with Leu911 and/or Lys941 of PI3Kα^{H1047R}, and wherein the first and second, second and third, and first and third hydrogen bonding moieties are each about 2.4 Å apart;
(ii) a hydrophobic moiety about 2.5 Å from the third hydrogen bonding moiety and is capable of interacting with Ile1022 of PI3Kα^{H1047R}; and
(iii) a first and a second aromatic moiety, wherein the first and second aromatic moieties together comprise a fused bicyclic aromatic ring system or two aromatic rings connected by a single bond, and wherein the first and second aromatic moieties are each capable of forming a pi-pi stacking interaction with Phe937 of PI3Kα^{H1047R};
(iv) a third aromatic moiety capable of forming a cation-pi interaction with Lys941 and/or a pi-pi stacking interaction with Tyr1021 and/or a of PI3Kα^{H1047R}; and
(v) a fourth hydrogen bonding moiety capable of forming hydrogen bonds with Glu950 and/or Arg949;
wherein the PI3Kα inhibitor has a K_{D} for PI3Kα^{H1047R} of about 0.1 nM to about 1 μM.

Some embodiments provide a PI3Kα inhibitor compound comprising:
(i) a first, a second, and a third hydrogen bonding moiety that each form one or more hydrogen bonds with Leu911 and/or Lys941 of PI3Kα^{H1047R}, and wherein the first and second, second and third, and first and third hydrogen bonding moieties are each about 2.4 Å apart;
(ii) a hydrophobic moiety about 2.5 Å from the third hydrogen bonding moiety interacts with Ile1022 of PI3Kα^{H1047R}; and
(iii) a first and a second aromatic moiety, wherein the first and second aromatic moieties together comprise a fused bicyclic aromatic ring system or two aromatic rings connected by a single bond, and wherein the first and second aromatic moieties each form a pi-pi stacking interaction with Phe937 of PI3Kα^{H1047R}.
(iv) a third aromatic moiety that forms a cation-pi interaction with Lys941 and/or a pi-pi stacking interaction with Tyr1021 and/or a of PI3Kα^{H1047R}; and
(v) a fourth hydrogen bonding moiety that forms one or more hydrogen bonds with Glu950 and/or Arg949;
wherein the PI3Kα inhibitor has a K_{D} for PI3Kα^{H1047R} of about 0.1 nM to about 1 μM.

In some embodiments, one of a first, a second, or a third hydrogen bonding moiety each capable of forming hydrogen bonds with Leu911 and/or Lys941 of PI3Kα^{H1047R} and hydrogen bond with the backbone carbonyl of Gly912.

In some embodiments, one of a first, a second, or a third hydrogen bonding moiety forms one or more hydrogen bonds with Leu9l 1 and/or Lys941 of PI3Kα^{H1047R} and forms a hydrogen bond with the backbone carbonyl of Gly912. In some embodiments, the first hydrogen bonding moiety forms one or more hydrogen bonds with Leu911 and/or Lys941 of PI3Kα^{H1047R} and forms a hydrogen bond with the backbone carbonyl of Gly912. In some embodiments, the second hydrogen bonding moiety forms one or more hydrogen bonds with Leu911 and/or Lys941 of PI3Kα^{H1047R} and forms a hydrogen bond with the backbone carbonyl of Gly912. In some embodiments, the third hydrogen bonding moiety forms one or more hydrogen bonds with Leu911 and/or Lys941 of PI3Kα^{H1047R} and forms a hydrogen bond with the backbone carbonyl of Gly912.

In some embodiments, the first, second, and third hydrogen bonding moieties are each independently selected from —C(═O)—, —CH(OH)—, —C(═NH)—, —CH(NH₂)—, —NH—, —O—, —S(═NH)(O)— and —S(═O)ₘ—; wherein m is 0, 1 or 2; and
wherein the first and second, and second and third hydrogen bonding moieties are not the same.

In some embodiments, the first hydrogen bonding moiety is —C(═O)—. In some embodiments, the first hydrogen bonding moiety is —CH(OH)—. In some embodiments, the first hydrogen bonding moiety is —C(═NH)—. In some embodiments, the first hydrogen bonding moiety —CH(NH₂)—. In some embodiments, the first hydrogen bonding moiety is —NH—. In some embodiments, the first hydrogen bonding moiety is —O—. In some embodiments, the first hydrogen bonding moiety is —S(═NH)(O)—. In some embodiments, the first hydrogen bonding moiety —S(═O)ₘ—.

In some embodiments, the second hydrogen bonding moiety is —C(═O)—. In some embodiments, the second hydrogen bonding moiety is —CH(OH)—. In some embodiments, the second hydrogen bonding moiety is —C(═NH)—. In some embodiments, the second hydrogen bonding moiety —CH(NH₂)—. In some embodiments, the second hydrogen bonding moiety is —NH—. In some embodiments, the second hydrogen bonding moiety is —O—. In some embodiments, the second hydrogen bonding moiety is —S(═NH)(O)—. In some embodiments, the second hydrogen bonding moiety —S(═O)ₘ—.

In some embodiments, the third hydrogen bonding moiety is —C(═O)—. In some embodiments, the third hydrogen bonding moiety is —CH(OH)—. In some embodiments, the third hydrogen bonding moiety is —C(═NH)—. In some embodiments, the third hydrogen bonding moiety —CH(NH₂)—. In some embodiments, the third hydrogen bonding moiety is —NH—. In some embodiments, the third hydrogen bonding moiety is —O—. In some embodiments, the third hydrogen bonding moiety is —S(═NH)(O)—. In some embodiments, the third hydrogen bonding moiety —S(═O)ₘ—.

In some embodiments, m is 0. In some embodiments, m is 1. In some embodiments, m is 2.

In some embodiments, the hydrophobic moiety is C1-C6 alkyl or C1-C6 haloalkyl. In some embodiments, the hydrophobic moiety is C1-C6 alkyl. In some embodiments, the hydrophobic moiety is C1-C6 haloalkyl. In some embodiments, the hydrophobic moiety is C1-C3 alkyl or C1-C3 haloalkyl. In some embodiments, the hydrophobic moiety is C1-C3 alkyl. In some embodiments, the hydrophobic moiety is C1-C3 haloalkyl. In some embodiments, the hydrophobic moiety is methyl. In some embodiments, the hydrophobic moiety is trifluoromethyl.

Some embodiments provide a PI3Kα inhibitor compound comprising a first and a second aromatic moiety joined by a linking group to a third aromatic moiety, wherein the first and a second aromatic moieties comprise a fused bicyclic aromatic ring system or two aromatic rings connected by a single bond, wherein:
(i) the linking group is capable of forming hydrogen bonds with Leu911 and/or Lys941 of PI3Kα^{H1047R}.
(ii) the first and second aromatic moieties are each capable of forming a pi-pi stacking interaction with Phe937 of PI3Kα^{H1047R} and
(iii) the third aromatic moiety is capable of forming an optional cation-pi interaction with Lys941 and/or a pi-pi stacking interaction with Tyr1021 of PI3Kα^{H1047R};
wherein the PI3Kα inhibitor has a K_{D} for PI3Kα^{H1047R} of about 0.1 nM to about 1 μM.

Some embodiments provide a PI3Kα inhibitor compound comprising a first and a second aromatic moiety joined by a linking group to a third aromatic moiety, wherein the first and a second aromatic moieties comprise a fused bicyclic aromatic ring system or two aromatic rings connected by a single bond, wherein:
(i) the linking group forms hydrogen bonds with Leu911 and/or Lys941 of PI3Kα^{H1047R};
(ii) the first and second aromatic moieties form a pi-pi stacking interaction with Phe937 of PI3Kα^{H1047R}; and
(iii) the third aromatic moiety forms an optional cation-pi interaction with Lys941 and/or a pi-pi stacking interaction with Tyr1021 of PI3Kα^{H1047R};
wherein the PI3Kα inhibitor has a K_{D} for PI3Kα^{H1047R} of about 0.1 nM to about 1 μM.

In some embodiments, the linking group is capable of forming water-mediated hydrogen bonds with Leu911 and/or Lys941 of PI3Kα^{H1047R}. In some embodiments, the linking group is capable of forming water-mediated hydrogen bonds with Leu911 of PI3Kα^{H1047R}. In some embodiments, the linking group is capable of forming water-mediated hydrogen bonds with Lys941 of PI3Kα^{H1047R}. In some embodiments, the linking group is capable of forming water-mediated hydrogen bonds with Leu911 and/or Lys941 of PI3Kα^{H1047R}.

In some embodiments, the linking group forms water-mediated hydrogen bonds with Leu911 and/or Lys941 of PI3Kα^{H1047R}. In some embodiments, the linking group forms water-mediated hydrogen bonds with Leu911 of PI3Kα^{H1047R}. In some embodiments, the linking group forms water-mediated hydrogen bonds with Lys941 of PI3Kα^{H1047R}. In some embodiments, the linking group forms water-mediated hydrogen bonds with Leu911 and/or Lys941 of PI3Kα^{H1047R}.

In some embodiments, the linking group comprises an amide, a urea, an imidazole, a benzimidazole, or a carbamate.

In some embodiments, the linking group comprises an amide. In some embodiments, the linking group comprises a urea. In some embodiments, the linking group comprises an imidazole. In some embodiments, the linking group comprises a benzimidazole. In some embodiments, the linking group comprises a carbamate.

In some embodiments, the linking group is an amide. In some embodiments, the linking group is a urea. In some embodiments, the linking group is an imidazole. In some embodiments, the linking group is a benzimidazole. In some embodiments, the linking group is a carbamate.

In some embodiments, the first and second aromatic moieties comprise a fused bicyclic aromatic ring system.

In some embodiments, the fused bicyclic aromatic ring system is a 9-10 membered aromatic ring system. In some embodiments, the first and second aromatic moieties form a benzimidazolyl or benzofuranyl.

In some embodiments, the third aromatic moiety is a 5-6 membered heteroaryl. In some embodiments, the third aromatic moiety is a pyrazolyl, oxazolyl, thiazolyl, pyridinyl, or pyrimidinyl.

In some embodiments, the third aromatic moiety is a 9-10 membered heteroaryl. In some embodiments, the third aromatic moiety is a 9 membered heteroaryl. In some embodiments, the third aromatic moiety is selected from benzimidazolyl, purinyl, indazolyl, and imidazopyridinyl.

In some embodiments, the third aromatic moiety is phenyl.

In some embodiments, the compound is capable of forming a direct binding interaction with an allosteric pocket on PI3Kα. In some embodiments, the compound forms a direct binding interaction with an allosteric pocket on PI3Kα.

In some embodiments, the allosteric pocket comprises three or more of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises three of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises four of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises five of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises six of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises seven of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises eight of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises nine of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises ten of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises eleven of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises twelve of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises thirteen of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises fourteen of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises fifteen of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises sixteen of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises seventeen of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises eighteen of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises nineteen of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises twenty of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises twenty-one of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the allosteric pocket comprises Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the compounds described herein form a direct binding interaction with three or more of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

In some embodiments, the compounds described herein (i.e, PI3Kα inhibitors) form a direct binding interaction with two or more residues in Table 1 and/or Table 2. In some embodiments, the compounds described herein (i.e, PI3Kα inhibitors) form a direct binding interaction with two to ten residues in Table 1 and/or Table 2. In some embodiments, the compounds described herein (i.e, PI3Kα inhibitors) form a direct binding interaction with 5 to 8 residues in Table 1 and/or Table 2.

In some embodiments, the backbone carbon atoms of Phe937 are displaced by about 4 Å to about 7 Å upon binding to PI3Kα, for example, about 4 Å, about 4.5 Å, about 5 Å, about 5.5 Å, about 6 Å, about 6.5 Å, or about 7 Å. In some embodiments, the backbone carbon atoms of Phe937 are displaced by about 4 Å. In some embodiments, the backbone carbon atoms of Phe937 are displaced by about 5 Å. In some embodiments, the backbone carbon atoms of Phe937 are displaced by about 6 Å. In some embodiments, the backbone carbon atoms of Phe937 are displaced by about 7 Å.

In some embodiments, the backbone carbon atoms of Leu911 are displaced by about 5 Å to about 6 Å upon binding to PI3Kα, for example, about 5 Å, about 5.25 Å, about 5.5 Å, about 5.75 Å, or about 6 Å. In some embodiments, the backbone carbon atoms of Leu911 are displaced by about 5 Å. In some embodiments, the backbone carbon atoms of Leu911 are displaced by about 6 Å.

In some embodiments, the compound has an IC₅₀ for PI3Kα^{H1047R} of about 0.1 nM to about 500 nM. For example, about 0.1 nM, about 0.5 nM, about 1 nM, about 2 nM, about 3 nM, about 4 nM, about 5 nM, about 10 nM, about 20 nM, about 50 nM, about 75 nM, about 100 nM, about 150 nM, about 200 nM, about 250 nM, about 300 nM, about 350 nM, about 400 nM, about 450 nM, or about 500 nM.

In some embodiments, the compound has an IC₅₀ for PI3Kα^{H1047X} of about 0.1 nM to about 500 nM, wherein X is any amino acid residue. For example, about 0.1 nM, about 0.5 nM, about 1 nM, about 2 nM, about 3 nM, about 4 nM, about 5 nM, about 10 nM, about 20 nM, about 50 nM, about 75 nM, about 100 nM, about 150 nM, about 200 nM, about 250 nM, about 300 nM, about 350 nM, about 400 nM, about 450 nM, or about 500 nM. In some embodiments, the compound has an IC₅₀ for PI3Kα^{H1047X} of about 0.1 nM to about 50 nM. For example, about 0.1 nM, about 0.5 nM, about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, or about 50 nM. In some embodiments, the compound has an IC₅₀ for PI3Kα^{H1047X} of about 0.1 nM to about 25 nM. For example, about 0.1 nM, about 0.25 nM, about 0.5 nM, about 0.75 nM, about 1 nM, about 2 nM, about 3 nM, about 4 nM, about 5 nM, about 6 nM, about 7 nM, about 8 nM, about 9 nM, about 10 nM, about 12 nM, about 15 nM, about 18 nM, about 20 nM, about 22 nM, or about 25 nM. In some embodiments, the compound has an IC₅₀ for PI3Kα^{H1047X} of about 0.1 nM to about 10 nM. For example, about 0.1 nM, about 0.2 nM, about 0.3 nM, about 0.4 nM, about 0.5 nM, about 0.6 nM, about 0.7 nM, about 0.8 nM, about 0.9 nM, about 1 nM, about 1.5 nM, about 2 nM, about 2.5 nM, about 3 nM, about 3.5 nM, about 4 nM, about 4.5 nM, about 5 nM, about 5.5 nM, about 6 nM, about 6.5 nM, about 7 nM, about 7.5 nM, about 8 nM, about 8.5 nM, about 9 nM, about 9.5 nM, or about 10 nM.

In some embodiments, the compound has an IC₅₀ for PI3Kα^{H1047R} of about 0.1 nM to about 100 nM. For example, about 0.1 nM, about 0.5 nM, about 1 nM, about 2 nM, about 3 nM, about 4 nM, about 5 nM, about 6 nM, about 7 nM, about 8 nM, about 9 nM, about 10 nM, about 20 nM, about 30 nM, about 40 nM, about 50 nM, about 60 nM, about 70 nM, about 80 nM, about 90 nM, or about 100 nM.

In some embodiments, the compound has an IC₅₀ for PI3Kα^{H1047R} of about 0.1 nM to about 50 nM. For example, about 0.1 nM, about 0.5 nM, about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, or about 50 nM.

In some embodiments, the compound has an IC₅₀ for PI3Kα^{H1047R} of about 0.1 nM to about 25 nM. For example, about 0.1 nM, about 0.25 nM, about 0.5 nM, about 0.75 nM, about 1 nM, about 2 nM, about 3 nM, about 4 nM, about 5 nM, about 6 nM, about 7 nM, about 8 nM, about 9 nM, about 10 nM, about 12 nM, about 15 nM, about 18 nM, about 20 nM, about 22 nM, or about 25 nM.

In some embodiments, the compound has an IC₅₀ for PI3Kα^{H1047R} of about 0.1 nM to about 10 nM. For example, about 0.1 nM, about 0.2 nM, about 0.3 nM, about 0.4 nM, about 0.5 nM, about 0.6 nM, about 0.7 nM, about 0.8 nM, about 0.9 nM, about 1 nM, about 1.5 nM, about 2 nM, about 2.5 nM, about 3 nM, about 3.5 nM, about 4 nM, about 4.5 nM, about 5 nM, about 5.5 nM, about 6 nM, about 6.5 nM, about 7 nM, about 7.5 nM, about 8 nM, about 8.5 nM, about 9 nM, about 9.5 nM, or about 10 nM.

In some embodiments, the compound has an IC₅₀ for PI3Kα^{E542K} of about 0.1 nM to about 500 nM. For example, about 0.1 nM, about 0.5 nM, about 1 nM, about 2 nM, about 3 nM, about 4 nM, about 5 nM, about 10 nM, about 20 nM, about 50 nM, about 75 nM, about 100 nM, about 150 nM, about 200 nM, about 250 nM, about 300 nM, about 350 nM, about 400 nM, about 450 nM, or about 500 nM.

In some embodiments, the compound has an IC₅₀ for PI3Kα^{E545K} of about 0.1 nM to about 500 nM. For example, about 0.1 nM, about 0.5 nM, about 1 nM, about 2 nM, about 3 nM, about 4 nM, about 5 nM, about 10 nM, about 20 nM, about 50 nM, about 75 nM, about 100 nM, about 150 nM, about 200 nM, about 250 nM, about 300 nM, about 350 nM, about 400 nM, about 450 nM, or about 500 nM.

In some embodiments, the compound has an IC₅₀ for PI3Kα^{M1043X} of about 0.1 nM to about 500 nM, wherein X is any amino acid residue. For example, about 0.1 nM, about 0.5 nM, about 1 nM, about 2 nM, about 3 nM, about 4 nM, about 5 nM, about 10 nM, about 20 nM, about 50 nM, about 75 nM, about 100 nM, about 150 nM, about 200 nM, about 250 nM, about 300 nM, about 350 nM, about 400 nM, about 450 nM, or about 500 nM.

In some embodiments, the compound has a molecule weight of about 275 Da to about 650 Da. For example, about 275 Da, about 300 Da, about 350 Da, about 400 Da, about 450 Da, about 500 Da, about 550 Da, about 600 Da, or about 650 Da.

In some embodiments, the compound has a molecule weight of about 300 Da to about 500 Da. For example, about 300 Da, about 325 Da, about 350 Da, about 375 Da, about 400 Da, about 425 Da, about 450 Da, about 475 Da, or about 500 Da.

In some embodiments, the compound has a molecule weight of about 350 Da to about 450 Da. For example, about 350 Da, about 360 Da, about 370 Da, about 380 Da, about 390 Da, about 400 Da, about 410 Da, about 420 Da, about 430 Da, about 440 Da, or about 450 Da.

In some embodiments, the compound has a eudysmic ratio of about 8 to about 500. For example, about 8, about 15, about 25, about 40, about 60, about 80, about 100, about 150, about 200, about 250, about 300, about 350, about 400, about 450, or about 500.

In some embodiments, the compound has a eudysmic ratio of about 8 to about 75. For example, about 8, about 10, about 15, about 20, about 30, about 40, about 50, about 60, about 70, or about 75.

In some embodiments, the compound has a eudysmic ratio of about 50 to about 200. For example, about 50, about 75, about 100, about 125, about 150, about 175, or about 200.

In some embodiments, the compound has a eudysmic ratio of about 150 to about 300. For example, about 150, about 175, about 200, about 225, about 250, about 275, or about 300.

In some embodiments, the compound has a eudysmic ratio of about 250 to about 500. For example, about 250, about 275, about 300, about 325, about 350, about 375, about 400, about 425, about 450, about 475, or about 500.

In some embodiments, the compound has a K_{D} for PI3Kα^{H1047R} of about 0.1 nM to about 50 nM. For example, about 0.1 nM, about 0.5 nM, about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, or about 50 nM.

In some embodiments, the compound has a K_{D} for PI3Kα^{H1047R} of about 0.1 nM to about 25 nM. For example, about 0.1 nM, about 0.25 nM, about 0.5 nM, about 0.75 nM, about 1 nM, about 2 nM, about 3 nM, about 4 nM, about 5 nM, about 6 nM, about 7 nM, about 8 nM, about 9 nM, about 10 nM, about 12 nM, about 15 nM, about 18 nM, about 20 nM, about 22 nM, or about 25 nM.

In some embodiments, the compound has a K_{D} for PI3Kα^{H1047R} of about 0.1 nM to about 10 nM. For example, about 0.1 nM, about 0.2 nM, about 0.3 nM, about 0.4 nM, about 0.5 nM, about 0.6 nM, about 0.7 nM, about 0.8 nM, about 0.9 nM, about 1 nM, about 1.5 nM, about 2 nM, about 2.5 nM, about 3 nM, about 3.5 nM, about 4 nM, about 4.5 nM, about 5 nM, about 5.5 nM, about 6 nM, about 6.5 nM, about 7 nM, about 7.5 nM, about 8 nM, about 8.5 nM, about 9 nM, about 9.5 nM, or about 10 nM

In some embodiments, the compound is about 1-fold to about 50-fold selective for PI3Kα^{H1047R} over wild type PI3Kα. For example, about 1-fold, about 5-fold, about 10-fold, about 15-fold, about 20-fold, about 25-fold, about 30-fold, about 35-fold, about 40-fold, about 45-fold, or about 50-fold.

In some embodiments, the compound is about 3-fold to about 10-fold selective for PI3Kα^{H1047R} over wild type PI3Kα. For example, about 1-fold, about 2-fold, about 3-fold, about 4-fold, about 5-fold, about 6-fold, about 7-fold, about 8-fold, about 9-fold, or about 10-fold.

In some embodiments, the compound is about 10-fold to about 50-fold selective for PI3Kα^{H1047R} over wild type PI3Kα. For example, about 10-fold, about 15-fold, about 20-fold, about 25-fold, about 30-fold, about 35-fold, about 40-fold, about 45-fold, or about 50-fold.

In some embodiments, the compound (i.e., a PI3Kα described herein) has the structure of Formula (I-A):

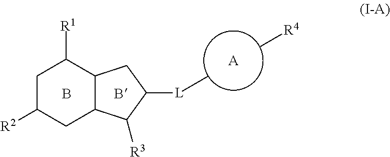

wherein:
Ring A is a 5-10 membered ring system substituted at a distal position with R⁴, wherein the ring system is selected from cycloalkyl, heterocyclyl, aryl, and heteroaryl;
Rings B and B′ together form a 9 membered ring system selected from cycloalkyl, heterocyclyl, aryl, and heteroaryl;
R¹ and R² are independently hydrogen, a hydrogen bond acceptor, or a hydrophobic moiety;
R³ is a hydrophobic moiety;
R⁴ comprises a hydrogen bond donor and/or a hydrogen bond acceptor; and
L comprises two or three groups selected from Z—C(═O)—, Z—CH(OH)—, Z—C(═NH)—, Z—CH(NH₂)—, Z—NH—, Z—O—, and Z—S(O)ₙ—, wherein n is 0, 1, or 2;
Z is absent, or is selected from the group consisting of C1-C3 alkyl, C1-C3 haloalkyl, and C3-C6 cycloalkyl; and
wherein no two adjacent groups in the L moiety are the same.

In some embodiments of Formula (I-A):
Ring A is a 5-10 membered ring system substituted at a distal position with R⁴, wherein the ring system is selected from cycloalkyl, heterocyclyl, aryl, and heteroaryl;
Rings B and B′ together form a 9 membered ring system selected from cycloalkyl, heterocyclyl, aryl, and heteroaryl;
R¹ and R² are independently hydrogen, a hydrogen bond acceptor, or a hydrophobic moiety;
R³ is a hydrophobic moiety;
R⁴ comprises a hydrogen bond donor and/or a hydrogen bond acceptor; and
Z is absent;
L comprises two or three groups selected from —C(═O)—, —CH(OH)—, —C(═NH)—, —CH(NH₂)—, —NH—, —O—, and —S(O)ₙ—; wherein n is 0, 1, or 2; and
wherein no two adjacent groups in the L moiety are the same.

In some embodiments of Formula (I-A):
Ring A is a 5-10 membered ring system substituted at a distal position with R⁴, wherein the ring system is selected from cycloalkyl, heterocyclyl, aryl, and heteroaryl;
Rings B and B′ together form a 9 membered ring system selected from cycloalkyl, heterocyclyl, aryl, and heteroaryl;
R¹ and R² are independently hydrogen, a hydrogen bond acceptor, or a hydrophobic moiety;
R³ is a hydrophobic moiety;
R⁴ comprises a hydrogen bond donor and/or a hydrogen bond acceptor; and
Z is absent;
L is two or three groups selected from —C(═O)—, —CH(OH)—, —C(═NH)—, —CH(NH₂)—, —NH—, —O—, and —S(O)ₙ—; wherein n is 0, 1, or 2; and
wherein no two adjacent groups in the L moiety are the same.

In some embodiments, Ring A is a 5-10 membered aryl substituted at a distal position with R⁴. In some embodiments, Ring A is phenyl substituted at the 4-position with R⁴.

In some embodiments, Ring A is a 5-10 membered cycloalkyl substituted at a distal position with R⁴. In some embodiments, Ring A 5-7 membered cycloalkyl substituted at a distal position with R⁴. In some embodiments, Ring A is cyclohexyl substituted at the 4-position with R⁴.

In some embodiments, Ring A is a 5-10 membered heterocyclyl substituted at a distal position with R⁴. In some embodiments, Ring A is a 5-7 membered heterocyclyl substituted at a distal position with R⁴. In some embodiments, Ring A is a 8-10 membered heterocyclyl substituted at a distal position with R⁴.

In some embodiments, Ring A is a 5-10 membered heteroaryl. In some embodiments, Ring A is a 5-6 membered heteroaryl substituted at a distal position with R⁴. In some embodiments, Ring A is a 9-10 membered heteroaryl substituted at a distal position with R⁴.

In some embodiments, Rings B and B′ together form a is cyclononyl. In some embodiments, Rings B and B′ together form a 9 membered heterocyclyl, for example, aznanyl, oxonanyl, diazonanyl, or oxazananyl. In some embodiments, Rings B and B′ together form a 9 membered heteroaryl, for example, indole, isoindole, indolizine, indazole, benzimidazole, azaindole, azaindazole, pyrazolopyrimidine, purine, benzofuran, isobenzofuran, benzothiophene, benzoxazole, benzoisoxazole, benzothiazole, or benzoisothiazole. In some embodiments, Rings B and B′ together form indazole, benzimidazole, azaindole, azaindazole, pyrazolopyrimidine, benzofuran, or benzoxazole.

In some embodiments, R¹ and R² are the same. In some embodiments, R¹ and R² are different.

In some embodiments, R¹ is hydrogen. In some embodiments, R¹ is a hydrogen bond acceptor. In some embodiments, R¹ is a hydrophobic moiety.

In some embodiments, R² is hydrogen. In some embodiments, R² is a hydrogen bond acceptor. In some embodiments, R² is a hydrophobic moiety.

In some embodiments, R³ is a hydrophobic moiety.

In some embodiments, the hydrophobic moiety of R¹, R², and R³ is independently C1-C6 alkyl or C1-C6 haloalkyl. In some embodiments, the hydrophobic moiety is C1-C6 alkyl. In some embodiments, the hydrophobic moiety is C1-C6 haloalkyl. In some embodiments, the hydrophobic moiety is C1-C3 alkyl or C1-C3 haloalkyl. In some embodiments, the hydrophobic moiety is C1-C3 alkyl. In some embodiments, the hydrophobic moiety is C1-C3 haloalkyl. In some embodiments, the hydrophobic moiety is methyl. In some embodiments, the hydrophobic moiety is trifluoromethyl.

In some embodiments, R⁴ comprises a hydrogen bond donor or a hydrogen bond acceptor.

In some embodiments, R⁴ comprises a hydrogen bond donor and a hydrogen bond acceptor.

In some embodiments, R⁴ comprises a hydrogen bond donor and not a hydrogen bond acceptor. In some embodiments, R⁴ comprises a hydrogen bond acceptor and not a hydrogen bond donor.

In some embodiments, R⁴ is hydroxyl. In some embodiments, R⁴ is an ether, for example, a C1-C6 alkoxy(C1-C6 alkyl)-. In some embodiments, R⁴ is amino. In some embodiments, R⁴ is a secondary amine, for example, a cyclic or acyclic secondary amine, such as a mono- or di-C1-C6 alkylamine, or a 4-10 membered heterocyclyl. In some embodiments, R⁴ is a perfluoro alkyl, for example, a C1-C6 fluoroalkyl such as trifluoromethyl. In some embodiments, R⁴ is —CO₂H. In some embodiments, R⁴ is an aldehyde. In some embodiments, R⁴ is a ketone, for example, —C(═O)C1-C6 alkyl. In some embodiments, R⁴ is an amide. In some embodiments, R⁴ is a carbamate. In some embodiments, R⁴ is a urea. In some embodiments, R⁴ is a sulfoxide. In some embodiments, R⁴ is a sulfonamide.

In some embodiments, Z is absent.

In some embodiments, Z is C1-C3 alkyl. In some embodiments, Z is C1-C3 haloalkyl. In some embodiments, Z is C3-C6 cycloalkyl.

In some embodiments, L comprises two groups selected from —C(═O)—, —CH(OH)—, —C(═NH)—, —CH(NH₂)—, —NH—, —O—, and —S(O)ₙ—. In some embodiments, L consists essentially of two groups selected from —C(═O)—, —CH(OH)—, —C(═NH)—, —CH(NH₂)—, —NH—, —O—, and —S(O)ₙ—. In some embodiments, L comprises an amide. In some embodiments, L is an amide.

In some embodiments, L comprises three groups selected from —C(═O)—, —CH(OH)—, —C(═NH)—, —CH(NH₂)—, —NH—, —O—, and —S(O)ₙ—. In some embodiments, L consists essentially of three groups selected from —C(═O)—, —CH(OH)—, —C(═NH)—, —CH(NH₂)—, —NH—, —O—, and —S(O)ₙ—. In some embodiments, L comprises an alpha hydroxy amide. In some embodiments, L is an alpha hydroxy amide.

In some embodiments, L is two groups selected from —C(═O)—, —CH(OH)—, —C(═NH)—, —CH(NH₂)—, —NH—, —O—, and —S(O)ₙ—. In some embodiments, L consists essentially of two groups selected from —C(═O)—, —CH(OH)—, —C(═NH)—, —CH(NH₂)—, —NH—, —O—, and —S(O)ₙ—. In some embodiments, L comprises an amide. In some embodiments, L is an amide.

In some embodiments, L is three groups selected from —C(═O)—, —CH(OH)—, —C(═NH)—, —CH(NH₂)—, —NH—, —O—, and —S(O)ₙ—. In some embodiments, L consists essentially of three groups selected from —C(═O)—, —CH(OH)—, —C(═NH)—, —CH(NH₂)—, —NH—, —O—, and —S(O)ₙ—. In some embodiments, L comprises an alpha hydroxy amide. In some embodiments, L is an alpha hydroxy amide. In some embodiments of L, no two adjacent groups in the L moiety are the same, for example, L contains no —O—O— or —N—N— bonds.

In some embodiments, n is 0. In some embodiments, n is 1. In some embodiments, n is 2.

In some embodiments, the compound of Formula (I) has the structure of Formula (I-B):

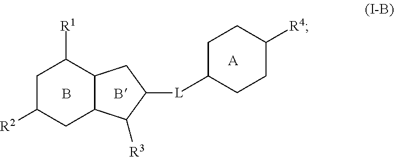

wherein Ring A is a phenyl, cyclohexyl, 6 membered heterocyclyl, or 6 membered heteroaryl.

In some embodiments, Ring A is phenyl. In some embodiments, Ring A is cyclohexyl. In some embodiments, Ring A is 6 membered heterocyclyl, for example, piperidinyl, piperazinyl, morpholinyl, or tetrahyrdopyranyl. In some embodiments, Ring A is 6 membered heteroaryl, for example, pyridinyl, pyrimidinyl, or pyridazinyl.

In some embodiments, L forms one or more hydrogen bonds with Leu911 and/or Lys941 of PI3Kα^{H1047R}. In some embodiments, L forms one hydrogen bond with Leu911 and/or Lys941 of PI3Kα^{H1047R}. In some embodiments, L forms two hydrogen bonds with Leu911 and/or Lys941 of PI3Kα^{H1047R}. In some embodiments, L forms three hydrogen bonds with Leu911 and/or Lys941 of PI3Kα^{H1047R}. In some embodiments, L forms four hydrogen bonds with Leu911 and/or Lys941 of PI3Kα^{H1047R}.

In some embodiments, the compound of Formula (I) has the structure of Formula (I-C):

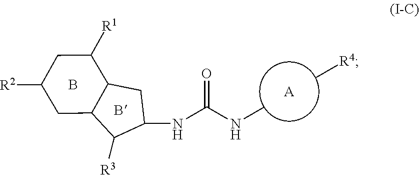

wherein Ring A is a 5-10 membered ring selected from aryl, cycloalkyl, heterocyclyl, and heteroaryl.

In some embodiments, Ring A is a 5-10 membered aryl. In some embodiments, Ring A is phenyl.

In some embodiments, Ring A is a 5-10 membered cycloalkyl. In some embodiments, Ring A 5-7 membered cycloalkyl. In some embodiments, Ring A is cyclohexyl.

In some embodiments, Ring A is a 5-10 membered heterocyclyl. In some embodiments, Ring A is a 5-7 membered heterocyclyl. In some embodiments, Ring A is a 8-10 membered heterocyclyl.

In some embodiments, Ring A is a 5-10 membered heteroaryl. In some embodiments, Ring A is a 5-6 membered heteroaryl. In some embodiments, Ring A is a 9-10 membered heteroaryl.

In some embodiments, the compound of Formula (I) has the structure of Formula (I-D):

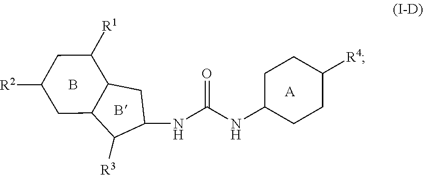

wherein Ring A is a phenyl, cyclohexyl, 6 membered heterocyclyl, or 6 membered heteroaryl.

In some embodiments, Ring A is phenyl. In some embodiments, Ring A is cyclohexyl. In some embodiments, Ring A is 6 membered heterocyclyl, for example, piperidinyl, piperazinyl, morpholinyl, or tetrahyrdopyranyl. In some embodiments, Ring A is 6 membered heteroaryl, for example, pyridinyl, pyrimidinyl, or pyridazinyl.

In some embodiments, the compound has a Log P value from about 1 to about 6. In some embodiments, the compound has a Log P value from about 1 to about 5. In some embodiments, the compound has a Log P value from about 1 to about 3. In some embodiments, the compound has a Log P value from about 1 to about 2. In some embodiments, the compound has a Log P value of about 1. In some embodiments, the compound has a Log P value of about 1.5. In some embodiments, the compound has a Log P value of about 2. In some embodiments, the compound has a Log P value of about 2.5. In some embodiments, the compound has a Log P value of about 3. In some embodiments, the compound has a Log P value of about 3.5. In some embodiments, the compound has a Log P value of about 4. In some embodiments, the compound has a Log P value of about 4.5. In some embodiments, the compound has a Log P value of about 5. In some embodiments, the compound has a Log P value of about 5.5. In some embodiments, the compound has a Log P value of about 6.

In some embodiments, Ring B and/or Ring B′ form a pi-pi stacking interaction with Phe937 of PI3Kα^{H1047R}. In some embodiments, Ring B and Ring B′ each form a pi-pi stacking interaction with Phe937 of PI3Kα^{H1047R}. In some embodiments, Ring B and not Ring B′ forms a pi-pi stacking interaction with Phe937 of PI3Kα^{H1047R}. In some embodiments, Ring B′ and not Ring B forms a pi-pi stacking interaction with Phe937 of PI3Kα^{H1047R}.

In some embodiments, Ring A is an aryl or heteroaryl ring which forms a cation-pi interaction with Lys941. In some embodiments, Ring A is an aryl ring which forms a cation-pi interaction with Lys941. In some embodiments, Ring A is a heteroaryl ring which forms a cation-pi interaction with Lys941.

In some embodiments, Ring A is an aryl or heteroaryl ring which forms a pi-pi stacking interaction with Tyr1021 of PI3Kα^{H1047R}. In some embodiments, Ring A is an aryl ring which forms a pi-pi stacking interaction with Tyr1021 of PI3Kα^{H1047R}. In some embodiments, Ring A is a heteroaryl ring which forms a pi-pi stacking interaction with Tyr1021 of PI3Kα^{H1047R}.

In some embodiments, the PI3Kα inhibitors described herein are inhibitors of mutant PI3Kα. In some embodiments, the PI3Kα inhibitors described herein are selective inhibitors of mutant PI3Kα over wild type PI3Kα. In some embodiments, the mutation in PI3Kα is selected from the group consisting of the mutations described in Table A, and combinations thereof.

TABLE A
PI3Kα Protein Amino Acid Substitutions/Insertions/Deletions^{A}

| Amino Acid Position | Non-Limiting Exemplary Mutations |
|---|---|
| 1 | M1 (Translation Start Site) |
| 4 | R4* (Nonsense Mutation) |
| 9 | E9G |
| 10 | L10_M16del |
| 11 | W11L, W11S, W11_P18del (In Frame Deletion) |
| 12 | G12D |
| 13 | I13T |
| 19 | R19I |
| 27 | P27T |
| 36 | C36Y |
| 38 | R38C, R38H, R38L, R38S |
| 39 | E39G, E39K |
| 57 | P57L |
| 65 | E65K |
| 66 | S66C |
| 69 | I69N |
| 71 | V71I |
| 75 | Q75E |
| 78 | E78* (nonsense mutation) |
| 80 | E80K |
| 81 | E81* (nonsense mutation), E81del (in |
| | frame deletion), E81K |
| 83 | F83L, F83S |
| 84 | D84H |
| 86 | T86S |
| 87 | R87T |
| 88 | R88Q |
| 90 | C90G, C90R, C90Y |
| 93 | R93P, R93Q, R93W |
| 102 | I102del |
| 103 | E103G, E103_G106delinsD (In Frame Deletion), |
| | E103_P104del (In Frame Deletion) |
| 104 | P104L, P104R, P104T |
| 105 | V105del, V105_R108del |
| 106 | G106D, G106R, G106S, G106V, G106_R108del |
| | (In Frame Deletion), G106_N107del |
| | (In Frame Deletion) |
| 107 | N107S |
| 108 | R108C, R108H, R108L |
| 109 | E109_I112delinsD (In Frame Deletion) |
| 110 | E110del |
| 111 | K111del, K111E, K111N, K111R, |
| | K111_L113del (In Frame Deletion) |
| 113 | L113del |
| 115 | R115L, R115P |
| 116 | E116K |
| 118 | G118D |
| 123 | M123I |
| 124 | P124A |
| 151 | V151M |
| 165 | Y165H |
| 170 | N170S |
| 182 | Y182H |
| 213 | H213N |
| 224 | A224S |
| 239 | L239R |
| 258 | D258N |
| 262 | L262I |
| 266 | P266T |
| 267 | L267M |
| 272 | Y272* (Nonsense Mutation) |
| 274 | R274K |
| 279 | L279I |
| 282 | M282V |
| 292 | S292I |
| 296 | Q296E |
| 300 | D300V |
| 310 | R310C |
| 322 | T322A |
| 335 | R335G |
| 337 | K337N |
| 339 | L339I |
| 342 | T342S |
| 344 | V344A, V344G, V344M |
| 345 | N345H, N345I, N345K, N345T, N345Y |
| 350 | D350G, D350N |
| 351 | I351T |
| 357 | R357Q |
| 359 | G359R |
| 363 | G363A |
| 364 | G364R |
| 365 | E365K, E365V |
| 366 | P366R |
| 378 | C378F, C378R, C378Y |
| 379 | S379T |
| 380 | N380S |
| 390 | D390N |
| 392 | Y392H |
| 398 | R398H |
| 399 | A399T |
| 401 | R401Q |
| 405 | S405F |
| 406 | I406V |
| 412 | R412Q |
| 417 | E417K |
| 418 | E418K |
| 420 | C420R |
| 432 | Y432C |
| 447 | P447_L455del (In frame Deletion) |
| 449 | P449L, P449S |
| 450 | H450_P458del (In Frame Deletion) |
| 451 | G451R, G451V, G451_D454del (In Frame |
| | Deletion) |
| 452 | L452_G460del (In Frame Deletion) |
| 453 | E453del, E453K, E453Q, E453_G460delinsDDF |
| | (in Frame Deletion), E453_L455del |
| 454 | D454Y |
| 455 | L455_G463del (In Frame Deletion) |
| 463 | G463_N465delinsD (In Frame Deletion) |
| 469 | E469A, E469delinsDK (In Frame Insertion) |
| 471 | P471A, P471L |
| 474 | E474A |
| 475 | L475F |
| 479 | W479* |
| 495 | H495L, H495Y |
| 499 | S499F |
| 519 | R519G |
| 520 | D520V |
| 522 | E522A |
| 531 | L531V |
| 539 | P539R, P539S |
| 542 | E542A, E542G, E542K, E542Q, E542V |
| 545 | E545A, E545D, E545G, E545K, E545Q |
| 546 | Q546E, Q546H, Q546K, Q546P, Q546R |
| 547 | E547D, E547K |
| 552 | W552C |
| 569 | L569I |
| 576 | S576Y |
| 581 | A581S |
| 589 | D589N |
| 600 | E600K, E600V |
| 603 | D603H |
| 604 | C604R |
| 606 | Y606C |
| 607 | P607Q |
| 609 | P609H |
| 614 | F614I |
| 617 | R617Q, R617W |
| 617 | R617W |
| 629 | S629C |
| 636 | V636L |
| 642 | E642K |
| 643 | Q643H |
| 658 | L658F |
| 667 | F667L |
| 673 | S673T |
| 674 | E674* (Nonsense mutation), E674D, E674Q |
| 682 | Q682K, Q682Rfs*18 (Frame Shift Deletion) |
| 683 | R683M |
| 684 | F684L |
| 693 | R693H |
| 710 | E710Q |
| 711 | K711N |
| 722 | E722K |
| 725 | D725G, D725N |
| 726 | E726K |
| 729 | K729N |
| 732 | M732I |
| 737 | E737K |
| 741 | R741Q |
| 744 | F744I |
| 746 | D746Y |
| 749 | Q749H |
| 752 | L752V |
| 766 | L766F |
| 770 | R770Q |
| 773 | S773F |
| 777 | R777M, R777K |
| 791 | E791Q |
| 811 | M811I |
| 816 | I816S |
| 818 | R818C, R818H |
| 849 | E849K |
| 852 | R852Q |
| 865 | G865D |
| 866 | L866F, L866W |
| 879 | Q879R |
| 886 | K886E |
| 901 | C901F |
| 903 | G903E |
| 905 | C905S |
| 909 | F909C |
| 914 | G914R |
| 929 | L929M |
| 930 | F930V |
| 939 | D939G |
| 948 | K948E |
| 951 | R951C |
| 953 | P953S |
| 956 | L956F |
| 958 | Q958R |
| 970 | E970K |
| 971 | C971R |
| 978 | E978K |
| 979 | R979G |
| 985 | Y985* |
| 989 | L989V |
| 992 | R992L, R992P |
| 997 | L997I |
| 1002 | F1002L |
| 1004 | M1004I, M1004R, M1004V |
| 1005 | M1005V |
| 1006 | L1006R |
| 1007 | G1007R |
| 1012 | E1012Q |
| 1015 | S1015Y |
| 1016 | F1016C |
| 1017 | D1017N |
| 1020 | A1020T |
| 1021 | Y1021C, Y1021H |
| 1025 | T1025A, T1025S |
| 1023 | R1023Q^{†} |
| 1026 | L1026I |
| 1029 | D1029H |
| 1037 | E1037K |
| 1040 | M1040I, M1040V |
| 1043 | M1043I, M1043L, M1043T, M1043V |
| 1044 | N1044I, N1044K, N1044Y |
| 1045 | D1045A, D1045V |
| 1047 | H1047L, H1047Q, H1047R, H1047Y |
| 1048 | H1048R |
| 1049 | G1049R |
| 1052 | T1052K |
| 1055 | M1055I |
| 1058 | I1058M |
| 1065 | H1065L |
| 1066 | A1066V |
| 1068 | N1068Y, N1068fs*5 (Frame Shift Insertion) |
| 1069 | *1069Wext*4 (nonstop Mutation) |

Unless noted otherwise, the mutations of Table A are found in cBioPortal database derived from Cerami et al., Cancer Discovery. May 2012 2; 401; and Gao et al., Sci. Signal. 6, p11 (2013). See also, Velho, et al., Eur J Cancer. 2005 July; 41(11):1649-54. doi: 10.1016/j.ejca.2005.04.022. PMID: 15994075.

In some embodiments, the PI3Kα described herein comprises one or more mutations as described in Table A. In some embodiments, the described herein PI3Kα described herein comprises one or more mutations in residues 1043-1069, as described in Table A. In some embodiments, the PI3Kα described herein comprises one or more mutations in residues 1043-1049, as described in Table A. In some embodiments, the PI3Kα described herein comprises one or more mutations in residues 1043-1045, as described in Table A. In some embodiments, the PI3Kα described herein comprises one or more mutations in residues 1045-1047, as described in Table A. In some embodiments, the PI3Kα described herein comprises one or more mutations in residues 1047-1049, as described in Table A. In some embodiments, the PI3Kα described herein comprises one or more mutations in residues 542-547, as described in Table A. In some embodiments, the PI3Kα described herein comprises one or more mutations in residue 542 or residue 545, as described in Table A.

In some embodiments, the PI3Kα described herein comprises one mutation as described in Table A. In some embodiments, the PI3Kα described herein comprises one mutation in residues 1043-1069, as described in Table A. In some embodiments, the PI3Kα described herein comprises one mutation in residues 1043-1049, as described in Table A. In some embodiments, the PI3Kα described herein comprises one mutation in residues 1043-1045, as described in Table A. In some embodiments, the PI3Kα described herein comprises one mutation in residues 1045-1047, as described in Table A. In some embodiments, the PI3Kα described herein comprises one mutation in residues 1047-1049, as described in Table A. In some embodiments, the PI3Kα described herein comprises one mutation in residues 542-547, as described in Table A. In some embodiments, the PI3Kα described herein comprises one mutation in residue 542 or residue 545, as described in Table A.

In some embodiments, the PI3Kα described herein is selected from the group consisting of E542A, E542G, E542K, E542Q, E542V, E545A, E545D, E545G, E545K, E545Q, M1043I, M1043L, M1043T, M1043V, H1047L, H1047Q, H1047R, H1047Y, G1049R, and combinations thereof. In some embodiments, the PI3Kα described herein is selected from the group consisting of E542A, E542G, E542K, E542Q, E542V, E545A, E545D, E545G, E545K, E545Q, M1043I, M1043L, M1043T, M1043V, H1047L, H1047Q, H1047R, H1047Y, G1049R.

In some embodiments, the PI3Kα described herein is H1047X, where X is any amino acid. In some embodiments, the PI3Kα described herein is E542X, where X is any amino acid. In some embodiments, the PI3Kα described herein is E545X, where X is any amino acid.

In some embodiments, the compounds described herein are not compounds disclosed in PCT Publication No. WO2021/222,556, which is hereby incorporated by reference for the purpose of excluding the compounds contained therein.

### Methods of Identifying PI3Kα Inhibitor Compounds

Provided herein are methods of identifying a PI3Kα inhibitor compound comprising:
(i) screening in silico a library for candidate compounds capable of forming a direct binding interaction with an allosteric pocket on PI3Kα, wherein a three-dimensional model of the binding site on PI3Kα is computationally derived from the atomic coordinates in Table 1; and
(ii) evaluating the candidate compounds identified in step (i) in one or more in vitro or in vivo assays for their ability to bind to the PI3Kα allosteric pocket to thereby identify the PI3Kα inhibitor;
wherein the PI3Kα inhibitor compound has a K_{D} for PI3Kα^{H1047R} of about 0.1 nM to about 1 μM.

Also provided herein are methods of identifying a PI3Kα inhibitor compound comprising:
(i) using the atomic coordinates in Table 2 to generate a 3-dimensional model of PI3Kα;
(ii) identifying three or more residues of an allosteric binding pocket;
(ii) generating a specific 3-dimensional target using the three or more allosteric binding pocket residues;
(iii) employing the specific 3-dimensional target to select a potential inhibitor of PI3Kα;
(iv) obtaining the potential inhibitor of PI3Kα; and
(v) contacting the potential inhibitor of PI3Kα with PI3Kα^{H1047R} in vitro to determine a K_{D} for PI3Kα^{H1047R}, wherein if the K_{D} for PI3Kα^{H1047R} is about 0.1 nM to about 1 μM, the potential inhibitor of PI3Kα is a PI3Kα^{H1047R} inhibitor.

Also provided herein are methods of identifying a PI3Kα inhibitor compound comprising:
(i) screening in silico a library for candidate compounds capable of forming a direct binding interaction with an allosteric pocket on PI3Kα, wherein a three-dimensional model of the binding site on PI3Kα is computationally derived from the atomic coordinates in Table 1 and Table 2; and
(ii) evaluating the candidate compounds identified in step (i) in one or more in vitro or in vivo assays for their ability to bind to the PI3Kα allosteric pocket to thereby identify the PI3Kα inhibitor;
wherein the PI3Kα inhibitor compound has a K_{D} for PI3Kα^{H1047R} of about 0.1 nM to about 1 μM.

Also provided herein are methods of identifying a PI3Kα inhibitor compound comprising:
(i) using the atomic coordinates in Table 1 and Table 2 to generate a 3-dimensional model of PI3Kα;
(ii) identifying three or more residues of an allosteric binding pocket;
(ii) generating a specific 3-dimensional target using the three or more allosteric binding pocket residues;
(iii) employing the specific 3-dimensional target to select a potential inhibitor of PI3Kα^{H1047R}.
(iv) obtaining the potential inhibitor of PI3Kα; and
(v) contacting the potential inhibitor of PI3Kα with PI3Kα^{H1047R} in vitro to determine a K_{D} for PI3Kα^{H047R} wherein if the K_{D} for PI3Kα^{H1047R} is about 0.1 nM to about 1 μM, the potential inhibitor of PI3KαL is a PI3Kα^{H1047R} inhibitor.

In some embodiments, the in vitro or in vivo assays are selected from an inhibition assay, a binding assay, or a probe displacement assay. In some embodiments the in vitro or in vivo assays are selected from the assays described in Examples 1 and 2.

TABLE 1

| | Atom | | | | | | | |
|---|---|---|---|---|---|---|---|---|
| Record | Serial | Atom | Residue | Chain | Residue | | | |
| Name | No. | Name | Name | Ident. | No. | X Coord. | Y Coord. | Z Coord. |
| ATOM | 1 | N | GLN | A | 809 | 43.068 | 37.182 | −26.702 |
| ATOM | 2 | CA | GLN | A | 809 | 42.417 | 37.272 | −25.396 |
| ATOM | 3 | C | GLN | A | 809 | 40.916 | 37.48 | −25.549 |
| ATOM | 4 | O | GLN | A | 809 | 40.152 | 36.925 | −24.763 |
| ATOM | 5 | CB | GLN | A | 809 | 43.029 | 38.392 | −24.559 |
| ATOM | 6 | CG | GLN | A | 809 | 42.429 | 38.488 | −23.15 |
| ATOM | 7 | CD | GLN | A | 809 | 43.12 | 39.509 | −22.273 |
| ATOM | 8 | OE1 | GLN | A | 809 | 44.26 | 39.924 | −22.501 |
| ATOM | 9 | NE2 | GLN | A | 809 | 42.434 | 39.957 | −21.25 |
| ATOM | 20 | N | LEU | A | 812 | 39.384 | 34.1 | −26.779 |
| ATOM | 21 | CA | LEU | A | 812 | 39.298 | 33.127 | −25.706 |
| ATOM | 22 | C | LEU | A | 812 | 38.194 | 33.498 | −24.694 |
| ATOM | 23 | O | LEU | A | 812 | 37.491 | 32.61 | −24.215 |
| ATOM | 24 | CB | LEU | A | 812 | 40.67 | 32.998 | −25.031 |
| ATOM | 25 | CG | LEU | A | 812 | 40.792 | 32.016 | −23.882 |
| ATOM | 26 | CD1 | LEU | A | 812 | 40.391 | 30.607 | −24.296 |
| ATOM | 27 | CD2 | LEU | A | 812 | 42.179 | 32.013 | −23.373 |
| ATOM | 40 | N | THR | A | 813 | 38.026 | 34.791 | −24.386 |
| ATOM | 41 | CA | THR | A | 813 | 36.994 | 35.228 | −23.455 |
| ATOM | 42 | C | THR | A | 813 | 35.632 | 34.988 | −24.066 |
| ATOM | 43 | O | THR | A | 813 | 34.739 | 34.485 | −23.385 |
| ATOM | 44 | CB | THR | A | 813 | 37.172 | 36.687 | −23.099 |
| ATOM | 45 | OG1 | THR | A | 813 | 38.507 | 36.881 | −22.655 |
| ATOM | 46 | CG2 | THR | A | 813 | 36.22 | 37.131 | −22.027 |
| ATOM | 55 | N | LEU | A | 911 | 35.567 | 35.19 | −14.021 |
| ATOM | 56 | CA | LEU | A | 911 | 36.755 | 35.497 | −14.818 |
| ATOM | 57 | C | LEU | A | 911 | 37.53 | 36.732 | −14.38 |
| ATOM | 58 | O | LEU | A | 911 | 38.603 | 36.983 | −14.926 |
| ATOM | 59 | CB | LEU | A | 911 | 36.352 | 35.665 | −16.286 |
| ATOM | 60 | CG | LEU | A | 911 | 35.596 | 34.515 | −16.924 |
| ATOM | 61 | CD1 | LEU | A | 911 | 35.299 | 34.836 | −18.365 |
| ATOM | 62 | CD2 | LEU | A | 911 | 36.37 | 33.199 | −16.823 |
| ATOM | 75 | N | GLY | A | 912 | 37.012 | 37.498 | −13.429 |
| ATOM | 76 | CA | GLY | A | 912 | 37.685 | 38.699 | −12.966 |
| ATOM | 77 | C | GLY | A | 912 | 37.749 | 39.785 | −14.02 |
| ATOM | 78 | O | GLY | A | 912 | 38.786 | 40.421 | −14.186 |
| ATOM | 83 | N | PHE | A | 937 | 41.338 | 43.444 | −19.399 |
| ATOM | 84 | CA | PHE | A | 937 | 40.973 | 43.196 | −17.994 |
| ATOM | 85 | C | PHE | A | 937 | 42.114 | 42.596 | −17.164 |
| ATOM | 86 | O | PHE | A | 937 | 42 | 42.603 | −15.938 |
| ATOM | 87 | CB | PHE | A | 937 | 39.684 | 42.372 | −17.837 |
| ATOM | 88 | CG | PHE | A | 937 | 39.72 | 40.932 | −18.278 |
| ATOM | 89 | CD1 | PHE | A | 937 | 39.367 | 40.578 | −19.564 |
| ATOM | 90 | CE1 | PHE | A | 937 | 39.349 | 39.256 | −19.949 |
| ATOM | 91 | CZ | PHE | A | 937 | 39.706 | 38.285 | −19.063 |
| ATOM | 92 | CD2 | PHE | A | 937 | 40.013 | 39.924 | −17.379 |
| ATOM | 93 | CE2 | PHE | A | 937 | 40.018 | 38.604 | −17.778 |
| ATOM | 104 | N | LEU | A | 938 | 43.207 | 42.112 | −17.797 |
| ATOM | 105 | CA | LEU | A | 938 | 44.34 | 41.532 | −17.065 |
| ATOM | 106 | C | LEU | A | 938 | 45.59 | 42.438 | −17.029 |
| ATOM | 107 | O | LEU | A | 938 | 46.657 | 41.987 | −16.596 |
| ATOM | 108 | CB | LEU | A | 938 | 44.727 | 40.214 | −17.728 |
| ATOM | 109 | CG | LEU | A | 938 | 43.67 | 39.158 | −17.786 |
| ATOM | 110 | CD1 | LEU | A | 938 | 44.131 | 38.02 | −18.618 |
| ATOM | 111 | CD2 | LEU | A | 938 | 43.324 | 38.668 | −16.428 |
| ATOM | 142 | N | LYS | A | 941 | 46.782 | 43.225 | −13.268 |
| ATOM | 143 | CA | LYS | A | 941 | 46.797 | 42.089 | −12.341 |
| ATOM | 144 | C | LYS | A | 941 | 48.166 | 41.961 | −11.663 |
| ATOM | 145 | O | LYS | A | 941 | 48.225 | 41.553 | −10.509 |
| ATOM | 146 | CB | LYS | A | 941 | 46.466 | 40.794 | −13.091 |
| ATOM | 147 | CG | LYS | A | 941 | 46.195 | 39.602 | −12.188 |
| ATOM | 148 | CD | LYS | A | 941 | 45.892 | 38.357 | −12.994 |
| ATOM | 149 | CE | LYS | A | 941 | 45.925 | 37.115 | −12.148 |
| ATOM | 150 | NZ | LYS | A | 941 | 45.713 | 35.887 | −12.955 |
| ATOM | 165 | N | GLU | A | 950 | 45.037 | 44.422 | −6.601 |
| ATOM | 166 | CA | GLU | A | 950 | 43.886 | 44.996 | −7.302 |
| ATOM | 167 | C | GLU | A | 950 | 42.816 | 43.909 | −7.497 |
| ATOM | 168 | O | GLU | A | 950 | 42.517 | 43.535 | −8.627 |
| ATOM | 169 | CB | GLU | A | 950 | 44.323 | 45.624 | −8.65 |
| ATOM | 170 | CG | GLU | A | 950 | 43.28 | 46.532 | −9.292 |
| ATOM | 171 | CD | GLU | A | 950 | 43.295 | 46.612 | −10.808 |
| ATOM | 172 | OE1 | GLU | A | 950 | 43.208 | 45.553 | −11.472 |
| ATOM | 173 | OE2 | GLU | A | 950 | 43.358 | 47.747 | −11.334 |
| ATOM | 181 | N | ARG | A | 951 | 42.306 | 43.349 | −6.384 |
| ATOM | 182 | CA | ARG | A | 951 | 41.299 | 42.279 | −6.411 |
| ATOM | 183 | C | ARG | A | 951 | 40.068 | 42.733 | −7.189 |
| ATOM | 184 | O | ARG | A | 951 | 39.608 | 43.847 | −6.962 |
| ATOM | 185 | CB | ARG | A | 951 | 40.899 | 41.894 | −4.97 |
| ATOM | 186 | CG | ARG | A | 951 | 39.74 | 40.884 | −4.853 |
| ATOM | 187 | CD | ARG | A | 951 | 39.376 | 40.58 | −3.402 |
| ATOM | 188 | NE | ARG | A | 951 | 38.903 | 41.766 | −2.681 |
| ATOM | 189 | CZ | ARG | A | 951 | 38.557 | 41.784 | −1.397 |
| ATOM | 190 | NH1 | ARG | A | 951 | 38.62 | 40.675 | −0.666 |
| ATOM | 191 | NH2 | ARG | A | 951 | 38.144 | 42.911 | −0.831 |
| ATOM | 205 | N | VAL | A | 952 | 39.58 | 41.911 | −8.135 |
| ATOM | 206 | CA | VAL | A | 952 | 38.403 | 42.286 | −8.911 |
| ATOM | 207 | C | VAL | A | 952 | 37.178 | 42.158 | −8.015 |
| ATOM | 208 | O | VAL | A | 952 | 36.803 | 41.036 | −7.673 |
| ATOM | 209 | CB | VAL | A | 952 | 38.257 | 41.412 | −10.177 |
| ATOM | 210 | CG1 | VAL | A | 952 | 36.908 | 41.654 | −10.855 |
| ATOM | 211 | CG2 | VAL | A | 952 | 39.408 | 41.663 | −11.145 |
| ATOM | 222 | N | PHE | A | 1002 | 41.776 | 26.614 | −20.282 |
| ATOM | 223 | CA | PHE | A | 1002 | 42.85 | 27.584 | −20.069 |
| ATOM | 224 | C | PHE | A | 1002 | 44.208 | 26.894 | −20.016 |
| ATOM | 225 | O | PHE | A | 1002 | 45.173 | 27.435 | −20.544 |
| ATOM | 226 | CB | PHE | A | 1002 | 42.636 | 28.384 | −18.782 |
| ATOM | 227 | CG | PHE | A | 1002 | 41.539 | 29.422 | −18.831 |
| ATOM | 228 | CD1 | PHE | A | 1002 | 41.591 | 30.461 | −19.743 |
| ATOM | 229 | CE1 | PHE | A | 1002 | 40.599 | 31.426 | −19.775 |
| ATOM | 230 | CZ | PHE | A | 1002 | 39.6 | 31.414 | −18.84 |
| ATOM | 231 | CD2 | PHE | A | 1002 | 40.545 | 29.447 | −17.873 |
| ATOM | 232 | CE2 | PHE | A | 1002 | 39.569 | 30.43 | −17.889 |
| ATOM | 244 | N | MET | A | 1010 | 50.311 | 33.686 | −25.93 |
| ATOM | 245 | CA | MET | A | 1010 | 49.578 | 34.708 | −25.203 |
| ATOM | 246 | C | MET | A | 1010 | 50.336 | 35.036 | −23.943 |
| ATOM | 247 | O | MET | A | 1010 | 50.55 | 34.137 | −23.139 |
| ATOM | 248 | CB | MET | A | 1010 | 48.166 | 34.216 | −24.841 |
| ATOM | 249 | CG | MET | A | 1010 | 47.278 | 34.005 | −26.049 |
| ATOM | 250 | SD | MET | A | 1010 | 45.776 | 33.106 | −25.652 |
| ATOM | 251 | CE | MET | A | 1010 | 44.872 | 34.357 | −24.791 |
| ATOM | 263 | N | GLU | A | 1012 | 49.362 | 36.644 | −21.432 |
| ATOM | 264 | CA | GLU | A | 1012 | 48.436 | 36.663 | −20.315 |
| ATOM | 265 | C | GLU | A | 1012 | 48.305 | 35.266 | −19.68 |
| ATOM | 266 | O | GLU | A | 1012 | 47.993 | 35.183 | −18.497 |
| ATOM | 267 | CB | GLU | A | 1012 | 47.062 | 37.162 | −20.792 |
| ATOM | 268 | CG | GLU | A | 1012 | 47.092 | 38.455 | −21.603 |
| ATOM | 269 | CD | GLU | A | 1012 | 47.409 | 38.354 | −23.091 |
| ATOM | 270 | OE1 | GLU | A | 1012 | 47.418 | 37.224 | −23.63 |
| ATOM | 271 | OE2 | GLU | A | 1012 | 47.657 | 39.411 | −23.717 |
| ATOM | 279 | N | LEU | A | 1013 | 48.524 | 34.185 | −20.467 |
| ATOM | 280 | CA | LEU | A | 1013 | 48.444 | 32.794 | −20.023 |
| ATOM | 281 | C | LEU | A | 1013 | 49.747 | 32.073 | −20.339 |
| ATOM | 282 | O | LEU | A | 1013 | 49.894 | 31.507 | −21.429 |
| ATOM | 283 | CB | LEU | A | 1013 | 47.293 | 32.093 | −20.75 |
| ATOM | 284 | CG | LEU | A | 1013 | 45.899 | 32.353 | −20.218 |
| ATOM | 285 | CD1 | LEU | A | 1013 | 44.893 | 31.996 | −21.244 |
| ATOM | 286 | CD2 | LEU | A | 1013 | 45.629 | 31.55 | −18.959 |
| ATOM | 299 | N | ASP | A | 1018 | 48.523 | 31.897 | −13.629 |
| ATOM | 300 | CA | ASP | A | 1018 | 47.611 | 32.86 | −14.252 |
| ATOM | 301 | C | ASP | A | 1018 | 46.145 | 32.357 | −14.197 |
| ATOM | 302 | O | ASP | A | 1018 | 45.226 | 33.173 | −14.194 |
| ATOM | 303 | CB | ASP | A | 1018 | 48.036 | 33.133 | −15.705 |
| ATOM | 304 | CG | ASP | A | 1018 | 49.455 | 33.655 | −15.846 |
| ATOM | 305 | OD1 | ASP | A | 1018 | 49.959 | 34.271 | −14.886 |
| ATOM | 306 | OD2 | ASP | A | 1018 | 50.062 | 33.445 | −16.92 |
| ATOM | 312 | N | ILE | A | 1019 | 45.934 | 31.029 | −14.115 |
| ATOM | 313 | CA | ILE | A | 1019 | 44.602 | 30.426 | −14.027 |
| ATOM | 314 | C | ILE | A | 1019 | 44.152 | 30.349 | −12.555 |
| ATOM | 315 | O | ILE | A | 1019 | 42.955 | 30.322 | −12.312 |
| ATOM | 316 | CB | ILE | A | 1019 | 44.561 | 28.998 | −14.664 |
| ATOM | 317 | CG1 | ILE | A | 1019 | 45.322 | 28.93 | −16.012 |
| ATOM | 318 | CG2 | ILE | A | 1019 | 43.107 | 28.521 | −14.838 |
| ATOM | 319 | CD1 | ILE | A | 1019 | 45.478 | 27.543 | −16.572 |
| ATOM | 332 | N | TYR | A | 1021 | 43.568 | 32.306 | −10.18 |
| ATOM | 333 | CA | TYR | A | 1021 | 42.512 | 33.225 | −9.753 |
| ATOM | 334 | C | TYR | A | 1021 | 41.098 | 32.608 | −9.902 |
| ATOM | 335 | O | TYR | A | 1021 | 40.247 | 32.855 | −9.045 |
| ATOM | 336 | CB | TYR | A | 1021 | 42.605 | 34.562 | −10.511 |
| ATOM | 337 | CG | TYR | A | 1021 | 41.524 | 35.551 | −10.121 |
| ATOM | 338 | CD1 | TYR | A | 1021 | 41.626 | 36.303 | −8.963 |
| ATOM | 339 | CE1 | TYR | A | 1021 | 40.628 | 37.195 | −8.589 |
| ATOM | 340 | CZ | TYR | A | 1021 | 39.506 | 37.343 | −9.377 |
| ATOM | 341 | OH | TYR | A | 1021 | 38.499 | 38.204 | −8.989 |
| ATOM | 342 | CE2 | TYR | A | 1021 | 39.382 | 36.594 | −10.533 |
| ATOM | 343 | CD2 | TYR | A | 1021 | 40.38 | 35.698 | −10.888 |
| ATOM | 354 | N | ILE | A | 1022 | 40.855 | 31.774 | −10.936 |
| ATOM | 355 | CA | ILE | A | 1022 | 39.534 | 31.15 | −11.114 |
| ATOM | 356 | C | ILE | A | 1022 | 39.162 | 30.249 | −9.93 |
| ATOM | 357 | O | ILE | A | 1022 | 37.977 | 30.056 | −9.686 |
| ATOM | 358 | CB | ILE | A | 1022 | 39.381 | 30.396 | −12.483 |
| ATOM | 359 | CG1 | ILE | A | 1022 | 37.898 | 30.318 | −12.918 |
| ATOM | 360 | CG2 | ILE | A | 1022 | 39.989 | 28.989 | −12.428 |
| ATOM | 361 | CD1 | ILE | A | 1022 | 37.687 | 29.869 | −14.317 |

TABLE 2

| | Atom | | | | | | | |
|---|---|---|---|---|---|---|---|---|
| Record | Serial | Atom | Residue | Chain | Residue | | | |
| Name | No. | Name | Name | Ident. | No. | X Coord. | Y Coord. | Z Coord. |
| ATOM | 79 | N | ILE | A | 816 | 34.803 | 31.323 | −23.855 |
| ATOM | 80 | CA | ILE | A | 816 | 34.399 | 30.949 | −22.503 |
| ATOM | 81 | C | ILE | A | 816 | 32.96 | 31.412 | −22.249 |
| ATOM | 82 | O | ILE | A | 816 | 32.176 | 30.647 | −21.709 |
| ATOM | 83 | CB | ILE | A | 816 | 35.365 | 31.541 | −21.453 |
| ATOM | 84 | CG1 | ILE | A | 816 | 36.815 | 31.036 | −21.626 |
| ATOM | 85 | CG2 | ILE | A | 816 | 34.854 | 31.265 | −20.055 |
| ATOM | 86 | CD1 | ILE | A | 816 | 36.998 | 29.557 | −21.584 |
| ATOM | 100 | N | ILE | A | 910 | 33.427 | 35.366 | −12.145 |
| ATOM | 101 | CA | ILE | A | 910 | 34.263 | 34.167 | −12.193 |
| ATOM | 102 | C | ILE | A | 910 | 35.596 | 34.425 | −12.911 |
| ATOM | 103 | O | ILE | A | 910 | 36.632 | 33.949 | −12.449 |
| ATOM | 104 | CB | ILE | A | 910 | 33.485 | 32.964 | −12.799 |
| ATOM | 105 | CG1 | ILE | A | 910 | 32.304 | 32.578 | −11.874 |
| ATOM | 106 | CG2 | ILE | A | 910 | 34.434 | 31.768 | −13.045 |
| ATOM | 107 | CD1 | ILE | A | 910 | 31.342 | 31.617 | −12.423 |
| ATOM | 146 | N | ILE | A | 913 | 36.656 | 39.996 | −14.767 |
| ATOM | 147 | CA | ILE | A | 913 | 36.598 | 41.048 | −15.778 |
| ATOM | 148 | C | ILE | A | 913 | 36.173 | 42.332 | −15.033 |
| ATOM | 149 | O | ILE | A | 913 | 34.999 | 42.699 | −15.024 |
| ATOM | 150 | CB | ILE | A | 913 | 35.648 | 40.676 | −16.947 |
| ATOM | 151 | CG1 | ILE | A | 913 | 36.136 | 39.395 | −17.642 |
| ATOM | 152 | CG2 | ILE | A | 913 | 35.573 | 41.829 | −17.949 |
| ATOM | 153 | CD1 | ILE | A | 913 | 35.191 | 38.741 | −18.576 |
| ATOM | 165 | N | GLY | A | 914 | 37.126 | 42.97 | −14.37 |
| ATOM | 166 | CA | GLY | A | 914 | 36.864 | 44.171 | −13.593 |
| ATOM | 167 | C | GLY | A | 914 | 36.922 | 45.473 | −14.364 |
| ATOM | 168 | O | GLY | A | 914 | 36.52 | 46.506 | −13.825 |
| ATOM | 214 | N | HIS | A | 940 | 46.26 | 45.207 | −15.215 |
| ATOM | 215 | CA | HIS | A | 940 | 46.654 | 45.619 | −13.873 |
| ATOM | 216 | C | HIS | A | 940 | 46.609 | 44.484 | −12.841 |
| ATOM | 217 | O | HIS | A | 940 | 46.477 | 44.756 | −11.65 |
| ATOM | 218 | CB | HIS | A | 940 | 45.777 | 46.8 | −13.428 |
| ATOM | 219 | CG | HIS | A | 940 | 45.713 | 47.914 | −14.431 |
| ATOM | 220 | ND1 | HIS | A | 940 | 46.844 | 48.628 | −14.797 |
| ATOM | 221 | CE1 | HIS | A | 940 | 46.44 | 49.515 | −15.692 |
| ATOM | 222 | NE2 | HIS | A | 940 | 45.13 | 49.411 | −15.919 |
| ATOM | 223 | CD2 | HIS | A | 940 | 44.654 | 48.398 | −15.118 |
| ATOM | 224 | H | HIS | A | 940 | 45.27 | 45.14 | −15.406 |
| ATOM | 225 | HA | HIS | A | 940 | 47.682 | 45.985 | −13.866 |
| ATOM | 226 | HB3 | HIS | A | 940 | 46.098 | 47.206 | −12.467 |
| ATOM | 227 | HB2 | HIS | A | 940 | 44.758 | 46.445 | −13.313 |
| ATOM | 228 | HD1 | HIS | A | 940 | 47.784 | 48.498 | −14.452 |
| ATOM | 229 | HE1 | HIS | A | 940 | 47.086 | 50.231 | −16.177 |
| ATOM | 230 | HD2 | HIS | A | 940 | 43.614 | 48.105 | −15.106 |
| ATOM | 231 | H | HIS | A | 940 | 46.619 | 45.867 | −15.89 |
| ATOM | 421 | N | ALA | A1020 | 45.09 | 30.269 | −11.583 | 1 |
| ATOM | 422 | CA | ALA | A1020 | 44.792 | 30.156 | −10.15 | 1 |
| ATOM | 423 | C | ALA | A1020 | 43.689 | 31.083 | −9.635 | 1 |
| ATOM | 424 | O | ALA | A1020 | 42.936 | 30.677 | −8.749 | 1 |
| ATOM | 425 | CB | ALA | A1020 | 46.058 | 30.362 | −9.337 | 1 |
| ATOM | 472 | N | LYS | A1024 | 39.836 | 31.013 | −6.739 | 1 |
| ATOM | 473 | CA | LYS | A1024 | 39.406 | 31.938 | −5.692 | 1 |
| ATOM | 474 | C | LYS | A1024 | 37.95 | 32.313 | −5.901 | 1 |
| ATOM | 475 | O | LYS | A1024 | 37.194 | 32.353 | −4.932 | 1 |
| ATOM | 476 | CB | LYS | A1024 | 40.228 | 33.244 | −5.7 | 1 |
| ATOM | 477 | CG | LYS | A1024 | 41.679 | 33.122 | −5.272 | 1 |
| ATOM | 478 | CD | LYS | A1024 | 42.274 | 34.517 | −5.029 | 1.00101.11 |
| ATOM | 479 | CE | LYS | A1024 | 43.785 | 34.536 | −5.038 | 1.00104.71 |
| ATOM | 480 | NZ | LYS | A1024 | 44.312 | 35.924 | −4.992 | 1.00106.72 |
| ATOM | 495 | N | THR | A1025 | 37.559 | 32.625 | −7.154 | 1 |
| ATOM | 496 | CA | THR | A1025 | 36.192 | 33.038 | −7.457 | 1 |
| ATOM | 497 | C | THR | A1025 | 35.245 | 31.854 | −7.403 | 1 |
| ATOM | 498 | O | THR | A1025 | 34.172 | 31.975 | −6.826 | 1 |
| ATOM | 499 | CB | THR | A1025 | 36.118 | 33.738 | −8.803 | 1 |
| ATOM | 500 | OG1 | THR | A1025 | 36.572 | 32.86 | −9.832 | 1 |
| ATOM | 501 | CG2 | THR | A1025 | 36.924 | 35.004 | −8.834 | 1 |

In some embodiments, the cartesian coordinates in Table 1 or 2 are used to virtually screen compounds for their ability to bind to PI3Kα as described herein. These compounds can include, for example, libraries of commercially available compounds, enumerated virtual combinatorial libraries, and/or virtual synthesizable compound collections (like Enamine REAL). The virtual screening can be carried out using a variety of software suites, for example, GLIDE, MOE, GOLD, FRED, OEDocking, AutoDOCK, and combinations thereof. In some embodiments, approaches that allow for receptor flexibility are also be utilized. Software that accounts for such receptor flexibility includes, but is not limited to, docking approaches like IFD (“Induced Fit Docking”), IFD-MD, docking plus molecular dynamics, and combinations of any of the foregoing.

In some embodiments, the protein-ligand interactions and/or compound descriptions and/or binding site coordinates described in Tables 1 and 2 are used to develop a pharmacophore model. Examples of software packages that can be used to prepare such a model include, but are not limited to, PHASE/GPU-PHASE, ROCS/FastROCS, MOE, BLAZE, and combinations thereof. In some embodiments, the methods described herein further comprise using the 3D coordinates from Table 1 and/or 2 to prioritize compounds based on known docket approaches. In some embodiments, the described compounds can be used to identify molecules of similar shape, for example, via ROCS/FastROCS.

### Pharmaceutical Compositions

In some embodiments, the compounds described herein, and pharmaceutically acceptable salts thereof, are administered as a pharmaceutical composition that includes the compound, or a pharmaceutically acceptable salt thereof, and one or more pharmaceutically acceptable excipients.

### Methods of Treatment

Provided herein are methods for inhibiting phosphatidylinositol 4,5-bisphosphate 3-kinase isoform alpha (PI3Kα), encoded by PIK3CA gene. For example, provided herein are inhibitors of PI3Kα useful for treating or preventing diseases or disorders associated with dysregulation of a PIK3CA gene, a PI3Kα protein, or the expression or activity or level of any of the same (i.e., a PI3Kα-associated disease or disorder), such as PIK3CA-related overgrowth syndromes ((PROS), see, e.g., Venot, et al., Nature, 558, 540-546 (2018)), brain disorders (e.g., as macrocephaly-capillary malformation (MCAP) and hemimegalencephaly), congenital lipomatous (e.g., overgrowth of vascular malformations), epidermal nevi and skeletal/spinal anomalies (e.g., CLOVES syndrome) and fibroadipose hyperplasia (FH), or cancer (e.g., PI3Kα-associated cancer).

A “PI3Kα inhibitor” or “PI3Kα inhibitor compound” as used herein includes any compound exhibiting PI3Kα inactivation activity (e.g., inhibiting or decreasing).

The ability of test compounds to act as inhibitors of PI3Kα may be demonstrated by assays known in the art. The activity of the compounds and compositions provided herein as PI3Kα inhibitors can be assayed in vitro, in vivo, or in a cell line. In vitro assays include assays that determine inhibition of the kinase. Alternate in vitro assays quantitate the ability of the inhibitor to bind to the protein kinase and can be measured either by radio labelling the compound prior to binding, isolating the compound/kinase complex and determining the amount of radio label bound, or by running a competition experiment where new compounds are incubated with the kinase bound to known radio ligands.

Potency of a PI3Kα inhibitor as provided herein can be determined by IC₅₀ value. A compound with a lower EC₅₀ value, as determined under substantially similar conditions, is a more potent inhibitor relative to a compound with a higher EC₅₀ value. In some embodiments, the substantially similar conditions comprise determining a PI3Kα-dependent phosphorylation level, in vitro or in vivo (e.g., in tumor cells, A594 cells, U2OS cells, A431 cells, Ba/F3 cells, or 3T3 cells expressing a wild type PI3Kα, a mutant PI3Kα, or a fragment of any thereof).

Potency of a PI3Kα inhibitor as provided herein can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining a PI3Kα-dependent phosphorylation level, in vitro or in vivo (e.g., in tumor cells, SKOV3, T47D, CAL33, BT20, HSC2, OAW42, NCI, HCC1954, NCIH1048, Detroit562, A594 cells, U2OS cells, A431 cells, A594 cells, U2OS cells, Ba/F3 cells, or 3T3 cells expressing a wild type PI3Kα, a mutant PI3Kα, or a fragment of any thereof).

The selectivity between wild type PI3Kα and PI3Kα containing one or more mutations as described herein can also be measured using in vitro assays such as surface plasmon resonance and fluorence-based binding assays, and cellular assays such as the levels of pAKT, a biomarker of PI3Kα activity, or proliferation assays where cell proliferation is dependent on mutant PI3Kα kinase activity.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

The term “PI3Kα-associated disease or disorder” as used herein refers to diseases or disorders associated with or having a dysregulation of a PIK3CA gene, a PI3Kα protein, or expression or activity, or level of any of the same. Non-limiting examples of PI3Kα-associated diseases or disorders are described herein.

The term “PI3Kα-associated cancer” as used herein refers to cancers associated with or having a dysregulation of a PIK3CA gene, a PI3Kα protein, or expression or activity, or level of any of the same. Non-limiting examples of PI3Kα-associated cancer are described herein.

The phrase “dysregulation of a PIK3CA gene, a PI3Kα protein, or the expression or activity or level of any of the same” refers to a genetic mutation that results in increased PI3Kα protein expression and/or increased PI3Kα protein activity. For example, a mutation in a PIK3CA gene that encodes a PI3Kα that is constitutively active or has increased activity as compared to a protein encoded by a PIK3CA gene that does not include the mutation.

Provided herein is a method of treating a disease or disorder (e.g., a PI3Kα-associated disease or disorder) in a subject in need of such treatment, the method comprising administering to the subject a therapeutically effective amount of a compound of Formula (I), or a pharmaceutically acceptable salt thereof, or a pharmaceutical composition thereof.

Also provided herein is a method of treating cancer (e.g., a PI3Kα-associated cancer) in a subject in need of such treatment, the method comprising administering to the subject a therapeutically effective amount of a compound of Formula (I), or a pharmaceutically acceptable salt thereof, or a pharmaceutical composition thereof.

In some embodiments, the cancer (e.g., PI3Kα-associated cancer) is selected from a hematological cancer and a solid tumor.

In some embodiments, the cancer (e.g., PI3Kα-associated cancer) is selected from breast cancer (including both HER2⁺ and HER2⁻ breast cancer, ER⁺ breast cancer, and triple negative breast cancer), endometrial cancer, lung cancer (including adenocarcinoma lung cancer and squamous cell lung carcinoma), esophageal squamous cell carcinoma, ovarian cancer, colorectal cancer, esophagastric adenocarcinoma, bladder cancer, head and neck cancer (including head and neck squamous cell cancers such as oropharyngeal squamous cell carcinoma), thyroid cancer, glioma, cervical cancer, lymphangioma, meningioma, melanoma (including uveal melanoma), kidney cancer, pancreatic neuroendocine neoplasms (pNETs), stomach cancer, esophageal cancer, acute myeloid leukemia, relapsed and refractory multiple myeloma, and pancreatic cancer.

In some embodiments, the cancer (e.g., PI3Kα-associated cancer) is selected from breast cancer (including both HER2⁺ and HER2⁻ breast cancer, ER⁺ breast cancer, and triple negative breast cancer), colon cancer, rectal cancer, colorectal cancer, ovarian cancer, lymphangioma, meningioma, head and neck squamous cell cancer (including oropharyngeal squamous cell carcinoma), melanoma (including uveal melanoma), kidney cancer, pancreatic neuroendocine neoplasms (pNETs), stomach cancer, esophageal cancer, acute myeloid leukemia, relapsed and refractory multiple myeloma, pancreatic cancer, lung cancer (including adenocarcinoma lung cancer and squamous cell lung carcinoma), and endometrial cancer.

In some embodiments, the cancer (e.g., PI3Kα-associated cancer) is selected from breast cancer, lung cancer, endometrial cancer, esophageal squamous cell carcinoma, ovarian cancer, colorectal cancer, esophagastric adenocarcinoma, bladder cancer, head and neck cancer, thyroid cancer, glioma, and cervical cancer.

In some embodiments, the PI3Kα-associated cancer is breast cancer. In some embodiments, the PI3Kα-associated cancer is colorectal cancer. In some embodiments, the PI3Kα-associated cancer is endometrial cancer. In some embodiments, the PI3Kα-associated cancer is lung cancer.

In some embodiments, the PI3Kα-associated cancer is selected from the group consisting of: Adrenocortical Carcinoma; Astrocytoma; Bladder Urothelial Carcinoma; Breast Invasive Carcinoma (NOS); Breast Invasive Ductal Carcinoma; Breast Invasive Lobular Carcinoma; Breast Invasive Mixed Mucinous Carcinoma; Cervical Squamous Cell Carcinoma; Colon Adenocarcinoma; Colorectal Cancer; Cutaneous Melanoma; Dedifferentiated Liposarcoma; Diffuse Type Stomach Adenocarcinoma; Esophageal Adenocarcinoma; Esophageal Squamous Cell Carcinoma; Glioblastoma Multiforme; Head and Neck Squamous Cell Carcinoma; Hepatocellular Carcinoma; Intestinal Type Stomach Adenocarcinoma; Intrahepatic Cholangiocarcinoma; Leiomyosarcoma; Lung Adenocarcinoma; Lung Squamous Cell Carcinoma; Mucinous Adenocarcinoma of the Colon and Rectum; Mucinous Carcinoma; Mucinous Stomach Adenocarcinoma; Oligoastrocytoma; Oligodendroglioma; Pancreatic Adenocarcinoma; Papillary Renal Cell Carcinoma; Papillary Stomach Adenocarcinoma; Papillary Thyroid Cancer; Pleural Mesothelioma, Biphasic Type; Prostate Adenocarcinoma; Rectal Adenocarcinoma; Renal Clear Cell Carcinoma; Seminoma; Serous Ovarian Cancer; Signet Ring Cell Carcinoma of the Stomach; Stomach Adenocarcinoma; Tubular Stomach Adenocarcinoma; Undifferentiated Pleomorphic Sarcoma/Malignant Fibrous Histiocytoma/High-Grade Spindle Cell Sarcoma; Uterine Carcinosarcoma/Uterine Malignant Mixed Mullerian Tumor; Uterine Endometrioid Carcinoma; Uterine Mixed Endometrial Carcinoma; and Uterine Serous Carcinoma/Uterine Papillary Serous Carcinoma.

Also provided is a method for inhibiting PI3Kα activity in a cell, comprising contacting the cell with a compound of Formula (I), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof, to a subject having a cell having aberrant PI3Kα activity. In some embodiments, the cell is a cancer cell. In some embodiments, the cancer cell is any cancer as described herein. In some embodiments, the cancer cell is a PI3Kα-associated cancer cell.

As used herein, the term “contacting” refers to the bringing together of indicated moieties in an in vitro system or an in vivo system. For example, “contacting” a PI3Kα protein with a compound provided herein includes the administration of a compound provided herein to an individual or subject, such as a human, having a PI3Kα protein, as well as, for example, introducing a compound provided herein into a sample containing a cellular or purified preparation containing the PI3Kα protein.

Also provided herein is a method of inhibiting cell proliferation, in vitro or in vivo, the method comprising contacting a cell with an effective amount of a compound of Formula (I), or a pharmaceutically acceptable salt thereof, or a pharmaceutical composition thereof as defined herein.

Further provided herein is a method of increase cell death, in vitro or in vivo, the method comprising contacting a cell with an effective amount of a compound of Formula (I), or a pharmaceutically acceptable salt thereof, or a pharmaceutical composition thereof as defined herein. Also provided herein is a method of increasing tumor cell death in a subject. The method comprises administering to the subject an effective compound of Formula (I), or a pharmaceutically acceptable salt thereof, in an amount effective to increase tumor cell death.

### EXAMPLES

### Example 1: Preparation of Compound A

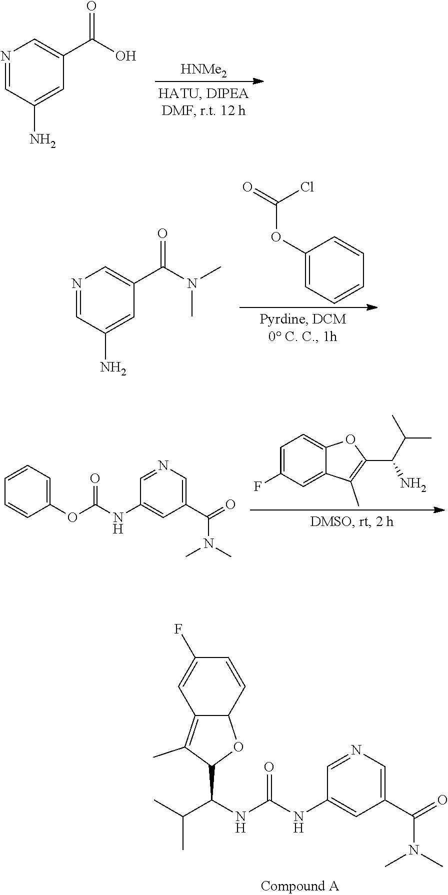

### Step 1

To a mixture of 5-aminonicotinic acid (0.77 g, 5.6 mmol), dimethylamine (5.6 mL, 11.2 mmol, 2 M solution THF), and DIPEA (3.6 g, 28.0 mmol) in DMF (35 mL) was added HATU (2.55 g, 6.72 mmol) at room temperature. The reaction mixture was stirred at room temperature for 12 h under N₂. After completion, the reaction was diluted with H₂O (30 mL), extracted with EtOAc (50 mL×3). The combined organic layers were washed with brine (40 mL), dried over Na₂SO₄, concentrated in vacuo to give the crude, which was purified with silica gel column chromatography, eluting with a gradient of 0-14% of MeOH in DCM to give 5-amino-N,N-dimethylnicotinamide (540 mg, yield: 58.4%) as a yellow solid. MS (ESI): mass calcd. C₈H₁₁N₃O, 165.09, m/z found 166.2 [M+H]⁺.

### Step 2

To a mixture of 5-amino-N,N-dimethylnicotinamide (50 mg, 0.3 mmol) and pyridine (47.4 mg, 0.6 mmol) in DCM (5 mL) was added phenyl carbonochloridate (52 mg, 0.33 mmol) at 0° C. The resulting reaction mixture was stirred at 0° C. for 1 h under N₂. After completion, the reaction was diluted with H₂O (30 mL), extracted with EtOAc (50 mL×3). The combined organic layers were washed with brine (40 mL), dried over Na₂SO₄, concentrated in vacuo to give the crude, which was purified with Prep-TLC (eluent: DCM/MeOH=20/1) to give phenyl (5-(dimethylcarbamoyl)pyridin-3-yl)carbamate (35 mg, yield: 41%) as a white solid. MS (ESI): mass calcd. C₁₅H₁₅N₃O₃, 285.11, m/z found 286.2 [M+H]⁺.

### Step 3

A solution of phenyl (5-(dimethylcarbamoyl)pyridin-3-yl)carbamate (35 mg, 0.28 mmol), (R)-1-(5-fluoro-3-methylbenzofuran-2-yl)-2-methylpropan-1-amine (Int-1) (73 mg, 0.33 mmol) in DMSO (5 mL) was stirred at room temperature for 2 h. After completion, the reaction was diluted with H₂O (30 mL), extracted with EtOAc (40 mL×3). The combined organic layers were washed with brine (40 mL), dried over Na₂SO₄, concentrated in vacuo to give the crude, which was purified with Prep-TLC (eluent: DCM/MeOH=10/1) to give (S)-5-(3-(1-(5-fluoro-3-methylbenzofuran-2-yl)-2-methylpropyl)ureido)-N,N-dimethylnicotinamide (36 mg, yield: 31%) as a white solid. MS (ESI): mass calcd. C₂₂H₂₅FN₄O₃, 412.19, m/z found 413.2 [M+H]⁺.

¹H NMR (400 MHz, dmso) δ 8.77 (s, 1H), 8.47 (d, J=2.5 Hz, 1H), 8.13 (d, J=1.9 Hz, 1H), 7.97-7.94 (m, 1H), 7.51 (dd, J=8.9, 4.1 Hz, 1H), 7.38 (dd, J=8.8, 2.7 Hz, 1H), 7.13-7.04 (m, 2H), 4.75 (t, J=8.6 Hz, 1H), 2.98 (s, 3H), 2.89 (s, 3H), 2.20 (s, 3H), 2.16-2.10 (m, 1H), 1.03 (d, J=6.7 Hz, 3H), 0.82 (d, J=6.7 Hz, 3H). Note: The chiral amine (used in the last step) is isolated via chiral HPLC.

### Preparation of Int-1 and Int-2 Chiral intermediates (R)- and (S)-1-(5-fluoro-3-methylbenzofuran-2-yl)-2-methylpropan-1-amine

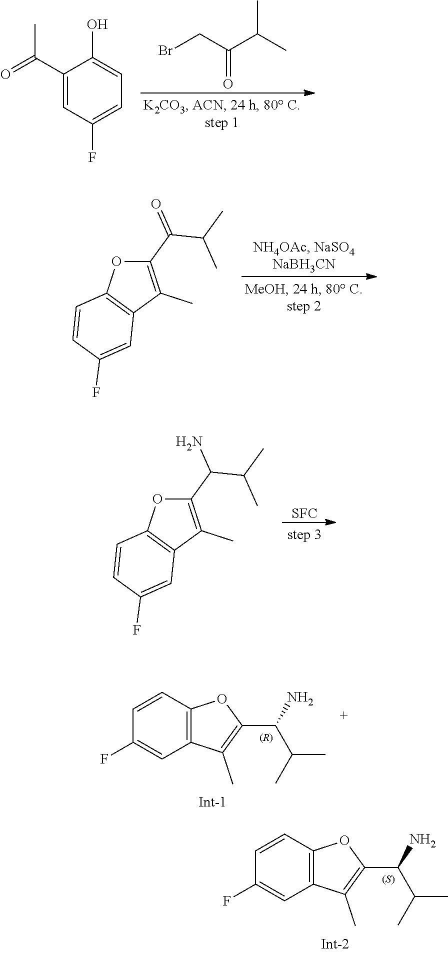

### Step 1

To a mixture of 1-(5-fluoro-2-hydroxyphenyl) ethan-1-one (18.6 g, 0.12 mol) and 1-bromo-3-methylbutan-2-one (20 g, 0.12 mol) in ACN (300 mL) was added K₂CO₃ (33.4 g, 0.24 mol). The reaction mixture was stirred at 80° C. for 24 h. After reaction, the reaction mixture was diluted with water and extracted with EA (500 mL×3). The combined organic layer was washed with brine and concentrated to give a residue which was purified by silica gel chromatography column (PE/EA from 0˜10%) to give 1-(5-fluoro-3-methylbenzofuran-2-yl)-2-methylpropan-1-one (24 g, 90%) as yellow oil. MS (ESI): mass calcd. for C₁₃H₁₃FO₂, 220.1, m/z found 221.2 [M+H]⁺.

### Step 2

A mixture of 1-(5-fluoro-3-methylbenzofuran-2-yl)-2-methylpropan-1-one (24 g, 0.11 mol), Na₂SO₄ (4.8 g) and NH₄OAc (83.9 g, 1.08 mol) in MeOH (400 mL) was stirred at room temperature for 1 h. NaBH₃CN (6.8 g, 0.10 mol) was added into the reaction mixture and the mixture was stirred at 80° C. for 24 h. After completion, the reaction mixture was filtered and the filtrate was concentrated to give a crude product which was re-dissolved in 10% NaOH aqueous solution. The aqueous solution was extracted with DCM (600 mL×3) and the combined organic layer was concentrated to give a residue which was purified by silica gel chromatography column (PE/EA from 0˜50%) to give racemic 1-(5-fluoro-3-methylbenzofuran-2-yl)-2-methylpropan-1-amine (16 g, 66%) as yellow solid. MS (ESI): mass calcd. for C₁₃H₁₆FNO, 221.1, m/z found 205.2 [M-NH₃+1]⁺.

### Step 3

Racemic 1-(5-fluoro-3-methylbenzofuran-2-yl)-2-methylpropan-1-amine (16 g) was separated by SFC to give (R)-1-(5-fluoro-3-methylbenzofuran-2-yl)-2-methylpropan-1-amine (Int-1) (peak 1, 7.1 g, 44%) and (S)-1-(5-fluoro-3-methylbenzofuran-2-yl)-2-methylpropan-1-amine (Int-2) (peak 2, 7.2 g, 45%) as yellow oil with the following chiral separation conditions. MS (ESI): mass calcd. for C₁₃H₁₆FNO, 221.1, m/z found 205.2.

Chiral Separation Conditions: Apparatus: SFC 150; Column: Daicel CHIRALCEL AS, 250 mm×30 mm I.D., 10 μm; Mobile phase: CO₂/MeOH [0.2% NH₃(7M Solution in MeOH)]=70/30; Flow rate: 80 g/min; Wavelength: UV 214 nm; Temperature: 35° C.

### (R)-1-(5-fluoro-3-methylbenzofuran-2-yl)-2-methylpropan-1-amine (Int-1) (Peak 1)

¹H NMR (400 MHz, DMSO) δ 7.49-7.46 (m, 1H), 7.34-7.31 (m, 1H), 7.08-7.01 (m, 1H), 3.69 (d, J=7.6 Hz, 1H), 2.22 (s, 2H), 2.15 (s, 3H), 1.96-1.87 (m, 1H), 1.00 (d, J=6.8 Hz, 3H), 0.74 (d, J=6.8 Hz, 3H).

### (S)-1-(5-fluoro-3-methylbenzofuran-2-yl)-2-methylpropan-1-amine (Int-2) (Peak 2)

¹H NMR (400 MHz, DMSO) δ 7.49-7.46 (m, 1H), 7.34-7.31 (m, 1H), 7.08-7.01 (m, 1H), 3.68 (d, J=7.6 Hz, 1H), 2.16 (s, 3H), 2.03-1.80 (m, 3H), 1.00 (d, J=6.8 Hz, 3H), 0.75 (d, J=6.8 Hz, 3H).

### Example 2: Preparation of Compound B

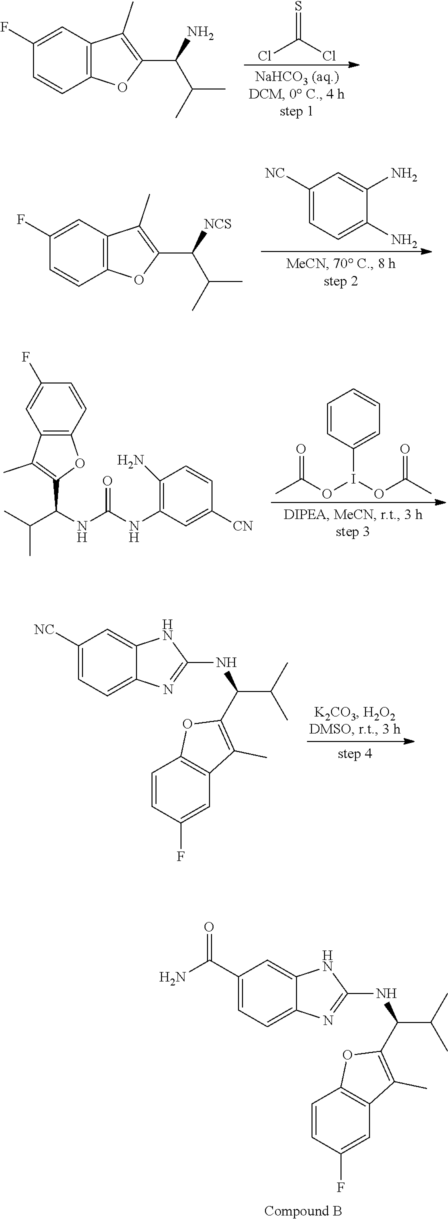

### Step 1

To a mixture of (S)-1-(5-fluoro-3-methylbenzofuran-2-yl)-2-methylpropan-1-amine (1.0 g, 4.52 mmol) and NaHCO₃ (sat. aq., 4 mL) in DCM (10 mL) was added thiophosgene (1.04 g, 9.05 mmol) at 0° C. The reaction mixture was stirred at 0° C. for 4 h. After completion, the resulting mixture was diluted with water (40 mL), extracted with DCM (40 mL×3). The combined organic layers were washed with brine (50 mL), dried over anhydrous Na₂SO₄, and filtered. The filtrate was concentrated under reduced pressure to afford (S)-5-fluoro-2-(1-isothiocyanato-2-methylpropyl)-3-methylbenzofuran (1.1 g, 93%) as a yellow oil, which was used into next step without further purification. ¹H NMR (400 MHz, DMSO-d₆) δ 7.62 (dd, J=9.0, 4.1 Hz, 1H), 7.45 (dd, J=8.7, 2.6 Hz, 1H), 7.21-7.15 (m, 1H), 5.24 (d, J=7.9 Hz, 1H), 2.38-2.29 (m, 1H), 2.23 (s, 3H), 1.10 (d, J=6.7 Hz, 3H), 0.86 (d, J=6.7 Hz, 3H).

### Step 2

A mixture of (S)-5-fluoro-2-(1-isothiocyanato-2-methylpropyl)-3-methylbenzofuran (200 mg, 0.76 mmol) and 3,4-diaminobenzonitrile (112 mg, 0.84 mmol) in MeCN (5 mL) was stirred at 70° C. for 8 h. After cooling to room temperature, the resulting mixture was concentrated under reduced pressure. The residue was purified by flash chromatography (DCM/MeOH from 1 to 5%) to afford (S)-1-(2-amino-5-cyanophenyl)-3-(1-(5-fluoro-3-methylbenzofuran-2-yl)-2-methylpropyl)thiourea (260 mg, 86%) as a yellow solid. MS (ESI): mass calcd. for C₂₁H₂₁FN₄OS, 396.14, m/z found 397.1 [M+H]⁺.

### Step 3

A solution of (S)-1-(2-amino-5-cyanophenyl)-3-(1-(5-fluoro-3-methylbenzofuran-2-yl)-2-methylpropyl)thiourea (260 mg, 0.66 mmol), phenyl-λ³-iodanediyl diacetate (318.8 mg, 0.99 mmol) and DIPEA (425.7 mg, 3.3 mmol) in MeCN (5 mL) was stirred at room temperature for 3 h. After completion, the resulting mixture was diluted with water (30 mL), extracted with DCM (30 mL×3). The combined organic layers were washed with brine (40 mL), dried over anhydrous Na₂SO₄, and filtered. The filtrate was concentrated under reduced pressure, and the residue was purified by flash chromatography (DCM/MeOH from 1 to 5%) to afford (S)-2-((1-(5-fluoro-3-methylbenzofuran-2-yl)-2-methylpropyl)amino)-1H-benzo[d]imidazole-6-carbonitrile (1, 144 mg, 58.9%) as an orange solid. MS (ESI): mass calcd. for C₂₁H₁₉FN₄O, 362.15, m/z found 363.2 [M+H]⁺. ¹H NMR (400 MHz, DMSO-d₆) δ 10.87 (d, J=37.8 Hz, 1H), 7.76 (m, 1H), 7.59-7.42 (m, 2H), 7.39-7.19 (m, 3H), 7.08 (m, 1H), 4.94 (q, J=8.9 Hz, 1H), 2.35-2.28 (m, 1H), 2.27 (s, 3H), 1.07 (d, J=6.6 Hz, 3H), 0.84 (d, J=6.7 Hz, 3H).

### Step 4

To a solution of (S)-2-((1-(5-fluoro-3-methylbenzofuran-2-yl)-2-methylpropyl)amino)-1H-benzo[d]imidazole-6-carbonitrile (50 mg 0.14 mmol), K₂CO₃ (38.6 mg, 0.28 mmol) and H₂O₂ (0.2 mL, 30% (aq)) in DMSO (2 mL) was stirred at room temperature for 3 h. The resulting mixture was diluted with water (10 mL), extracted with DCM (10 mL×3), and the combined organic layers were washed with brine (30 mL), dried over anhydrous Na₂SO₄, and filtered. The filtrate was concentrated under reduced pressure and the residue was purified by flash chromatography (DCM/MeOH from 1 to 8%) to afford (S)-2-((1-(5-fluoro-3-methylbenzofuran-2-yl)-2-methylpropyl)amino)-1H-benzo[d]imidazole-6-carboxamide (2, 30 mg, 56%) as a white solid. MS (ESI): mass calcd. for C₂₁H₂₁FN₄O₂, 380.16, m/z found 381.1 [M+H]⁺. ¹H NMR (400 MHz, DMSO-d₆) δ 10.62 (s, 1H), 7.68 (d, J=13.1 Hz, 2H), 7.51-7.33 (m, 4H), 7.14-6.97 (m, 3H), 4.93 (q, J=8.7 Hz, 1H), 2.34-2.31 (m, 1H), 2.28 (s, 3H), 1.08 (d, J=6.5 Hz, 3H), 0.84 (d, J=6.7 Hz, 3H).

### Example 3: Surface Plasmon Resonance (SPR)

SPR experiments were performed on a Biacore 8K instrument. A biotinylated recombinant PI3Kα H1047R protein was used in this study. The protein contained a full-length p110-α subunit harboring the H1047R mutation with an N-terminal AviTag, complexed with a truncated p85-α subunit (amino acid residues 322-694). The protein was first incubated with 1 μM wortmannin for 30 min at RT to covalently block the ATP binding site, then immobilized onto a streptavidin sensor chip by flowing the protein through the sensor chip at typically 20 μg/mL concentration and 2 μL/min flow rate for 1200 seconds. Compound binding affinities were measured in the multi-cycle kinetics mode, at 90 μL/min flow rate with 90 seconds association time and 240 seconds dissociation time. The running buffer contained 50 mM Tris, pH 7.5, 150 mMNaCl, 0.01% Brij35, 1 mM DTT, 1 mM MgCl₂, 0.05% Tween-20 and 2% DMSO. Temperature was maintained at 25° C. during experiments. SPR can also be utilized to study competition between Compounds A and B, i.e., by measuring the binding of Compound A in the presence and absence of Compound B, and by measuring the binding of Compound B in the presence and absence of Compound A.

The PI3Kα^{H1047R}+wortmannin K_{D} for Compound A is 38.4 nM and the PI3Kα^{H1047R}+wortmannin K_{D} for Compound B is 8.6 nM.

### Example 4: Homogenous Time-Resolved Fluorescence (HTRF)—pAKT-T47D

Compounds A and B were assayed using homogeneous time-resolved fluorescence (HTRF). Compound A has a pAKT IC₅₀ of 455 nM and Compound B has a pAKT IC₅₀ of 213 nM.

### Materials, Reagents, and Equipment

Gibco RPMI 1640 Medium, no phenol red; Gibco RPMI 1640 Medium; Gibco Trypsin-EDTA (0.5%), no phenol red; Gibco DPBS; Trypan blue solution 0.4% (Corning); Avantor Seradigm Premium Grade Fetal Bovine Serum (FBS); Greiner 784080-384 well TC treated white plates; pAKT (Ser473) HTRF; Gibco Insulin, human recombinant, zinc solution; Gibco Recovery Cell Culture Freezing Medium; Countess II FL Automated Cell Counter (ThermoFisher); Countess II Slides (ThermoFisher); Microscope; and PHERAstar FSX Microplate Reader (BMG LABTECH, Inc.).

### Procedure

The cell line ID was T47D.1, the HTRF detection was pAKT (S473), a PI3Kα H1047R mutation was present, the seeding density was 5000, the timepoint was 1 hour, and the medium used was RPMI+10% FBS (no phenol red)+0.2 units/mL bovine insulin.

### Cell Culture Maintenance:

The cell density was not permitted to reach 100% confluence. The cells were split 1.5 when they reached ˜80% confluence.
   Cells were split twice a week (Mon and Fri).
   Cells over passage 18 were not used (˜2 months of maintenance).
   Antibiotics were not used for tissue culture maintenance or assays.

### For Freezing Cells:

1. Trypsinized cells were collected and counted. Cells were pelleted at 1000 rpm, 5 minutes and supernatant was aspirated.
2. Pelleted cells were gently resuspended at 3e6 cells/1 mL of freezing medium (Gibco Freezing Medium). For example, if there were 9×10⁶ total cells, cell pellet was resuspended in 3 mL of freezing medium.
3. Measured aliquot of 1 mL of resuspended cells/cryovial. Cells were frozen in appropriate cell freezing container (i.e. Mr. Frosty or Corning CoolCell Freezing System) at −80° C.
4. Cells were transferred to Liquid Nitrogen Cryotank for long term storage.

### For Thawing Cells:

1. Cells were removed from liquid nitrogen tank. Cryovials were thawed in 37° C. waterbath until small “ice pellet” remained. This was then sprayed down with 70% ethanol before moving to TC/BSC hood.
2. Added 9 ml of fresh media to a 15 mL conical tube. Added 10 mL of fresh media to a T75 TC treated flask.
3. Gently transferred 1 mL of cells in freezing medium from cryovial to 15 mL conical tube containing media.
4. Centrifuged at 1000 rpm, 5 mins to pellet cells.
5. Aspirated media/freezing media.
6. Gently resuspended cell pellet in 5 mL fresh media and transferred to T75 flask with 10 mLs of fresh media. Place flasks in 37° C. incubator, 5% CO₂.

### Protocol

### Day 1

The procedure was as follows:
1. Prepared ARP:
   a. Stamped 12.5 nL from 10 mM source plate to destination plate using Echo. Sealed plate immediately and froze at −20° C. if it was not used on the same day.
   b. If a frozen ARP was used, the plate was thawed and spun at 1000 rpm×1 min.
2. Preparation of cells (adherent):
   a. Aspirated media from cells. Washed cells with sterile 1×PBS. Aspirated PBS and added appropriate amount of Trypsin.
   b. Once cells were fully trypsinized, added appropriate media to resuspend cells. Transferred cells to a 15 mL or 50 mL conical tube.
   c. Counted cells on the Countess II Cell Counter.
3. Plating of cells:
   a. Prepared cells at appropriate plating density. Dispensed 12 μL of diluted cells per well of a Greiner 784080-384 well TC treated white plate using a Multidrop Combi to columns 1-23. Added 12 μL of appropriate phenol free media only to column 24.
   b. Placed plates in 37° C. tissue culture incubator for appropriate treatment time.
4. Prepared HTRF Lysis Buffer
   a. Calculate the amount of HTRF lysis buffer master mix needed to perform the desired experiments plus any extra dead volume required for dispensing (4 μL required per well). Dilute the Blocking Reagent into 4× Lysis Buffer at a ratio of 1:25 (i.e. 0.1 mL Blocking Reagent Solution plus 2.4 mL 4× Lysis Buffer).
   b. Add 4 uL Lysis buffer master mix to all wells with sample or DMSO. Centrifuge the plates for 1 minute at 1000 rpm.
   c. Incubate at room temperature for 30 minutes.
5. Prepared HTRF Antibody
   a. Calculated the amount of HTRF antibody master mix needed to perform the desired experiments plus any extra dead volume required for dispensing (4 mL required per well). Eu Cryptate antibody and d2 antibody were added to detection buffer each at a ratio of 1:40 (i.e. 100 μL Eu Cryptate+100 μL d2 Cryptate+3800 μL detection buffer).
   b. 4 μL of antibody master mix was added to each well including the media only column 24.
   c. Centrifuged the plates for 1 minute at 1000 rpm. Placed lid on and created a “humidity chamber” by placing the plates into a ziplock bag with wet paper towels or something similar and incubated overnight at room temperature, keeping away from light.

### Day 2

6. Measured on the PHERAstar/Envision using the HTRF protocol. When plates were read, all wells were read.

The T47D pAKT IC₅₀ (nM) for Compound A is 598 nM and the T47D pAKT IC₅₀ (nM) for Compound B is 213 nM.

### Example 5: Virtual Screening

The cartesian coordinates in Table 1 or 2 are used to evaluate libraries of commercially available compounds, enumerated virtual combinatorial libraries, and/or virtual synthesizable compound collections (like Enamine REAL) using docking software. Examples of such software includes, but is not limited to GLIDE, MOE, GOLD, FRED, OEDocking, and AutoDOCK. Approaches that allow for receptor flexibility can also be utilized to account for required side chain adjustment. Such approaches include, but are not limited to IFD, IFD-MD, docking plus molecular dynamics, or a combination of any of the foregoing.

The protein-ligand interactions, compound descriptions, and/or binding site coordinates described in Tables 1 and 2 are used to develop a pharmacophore model to prioritize compounds from libraries of commercially available compounds, enumerated virtual combinatorial libraries, and/or virtual synthesizable compound collections (like Enamine REAL). Examples of such software packages include, but are not limited to PHASE/GPU-PHASE, ROCS/FastROCS, MOE, and BLAZE. In a second phase, the 3D coordinates from Table 1 or 2 are utilized to further prioritize using known docking approaches.

What is claimed is:

1. A PI3Kα inhibitor comprising a compound capable of forming a direct binding interaction with an allosteric pocket on PI3Kα revealed by displacing Phe937 and Leu938, thereby exposing the allosteric pocket;
wherein the allosteric pocket comprises Thr813, Leu911, and Phe1002;
wherein the compound forms direct binding interactions with one or more amino acids of the allosteric pocket; and
wherein the compound has an K_{D} for PI3Kα^{H1047R} of about 0.1 nM to about 1 μM.

2. The PI3Kα inhibitor of claim 1, wherein the allosteric pocket further comprises Phe937.

3. A PI3Kα inhibitor comprising a compound capable of forming a direct binding interaction with an allosteric pocket on PI3Kα;
wherein the allosteric pocket comprises Leu911, Phe937, and Phe1002;
wherein the compound forms direct binding interactions with one or more amino acids of the allosteric pocket; and
wherein the compound has an K_{D} for PI3Kα^{H1047R} of about 0.1 nM to about 1 μM.

4. The PI3Kα inhibitor of claim 3, wherein the allosteric pocket further comprises Thr813.

5. The PI3Kα inhibitor of any one of claims 1-4, wherein the allosteric pocket further comprises Lys941.

6. The PI3Kα inhibitor of any one of claims 1-5, wherein the allosteric pocket further comprises one to five amino acids selected from: Arg949, Glu950, Val952, Tyr1021, and Ile1022.

7. The PI3Kα inhibitor of any one of claims 1-6, wherein the allosteric pocket further comprises one of Arg949, Glu950, Val952, Tyr1021, and Ile1022.

8. The PI3Kα inhibitor of any one of claims 1-7, wherein the allosteric pocket further comprises Arg949.

9. The PI3Kα inhibitor of any one of claims 1-8, wherein the allosteric pocket further comprises Glu950.

10. The PI3Kα inhibitor of any one of claims 1-9, wherein the allosteric pocket further comprises Val952.

11. The PI3Kα inhibitor of any one of claims 1-10, wherein the allosteric pocket further comprises Tyr1021.

12. The PI3Kα inhibitor of any one of claims 1-11, wherein the allosteric pocket further comprises Ile1022.

13. The PI3Kα inhibitor of any one of claims 1-6, wherein the allosteric pocket further comprises two of Arg949, Glu950, Val952, Tyr1021, and Ile1022.

14. The PI3Kα inhibitor of any one of claims 1-6 or 13, wherein the allosteric pocket further comprises Arg949 and Glu950.

15. The PI3Kα inhibitor of any one of claims 1-6 or 13, wherein the allosteric pocket further comprises Arg949 and Val952.

16. The PI3Kα inhibitor of any one of claims 1-6 or 13, wherein the allosteric pocket further comprises Arg949 and Tyr1021.

17. The PI3Kα inhibitor of any one of claims 1-6 or 13, wherein the allosteric pocket further comprises Arg949 and Ile1022.

18. The PI3Kα inhibitor of any one of claims 1-6 or 13, wherein the allosteric pocket further comprises Glu950 and Val952.

19. The PI3Kα inhibitor of any one of claims 1-6 or 13, wherein the allosteric pocket further comprises Glu950 and Tyr1021.

20. The PI3Kα inhibitor of any one of claims 1-6 or 13, wherein the allosteric pocket further comprises Glu950 and Ile1022.

21. The PI3Kα inhibitor of any one of claims 1-6 or 13, wherein the allosteric pocket further comprises Val952 and Tyr1021.

22. The PI3Kα inhibitor of any one of claims 1-6 or 13, wherein the allosteric pocket further comprises Val952 and Ile1022.

23. The PI3Kα inhibitor of any one of claims 1-6 or 13, wherein the allosteric pocket further comprises Tyr1021 and Ile1022.

24. The PI3Kα inhibitor of any one of claims 1-6, wherein the allosteric pocket further comprises three of Arg949, Glu950, Val952, Tyr1021, and Ile1022.

25. The PI3Kα inhibitor of any one of claims 1-6 or 24, wherein the allosteric pocket further comprises Arg949, Glu950, and Val952.

26. The PI3Kα inhibitor of any one of claims 1-6 or 24, wherein the allosteric pocket further comprises Glu950, Val952, and Tyr1021.

27. The PI3Kα inhibitor of any one of claims 1-6 or 24, wherein the allosteric pocket further comprises Val952, Tyr1021, and Ile1022.

28. The PI3Kα inhibitor of any one of claims 1-6 or 24, wherein the allosteric pocket further comprises Arg949, Val952, and Tyr1021.

29. The PI3Kα inhibitor of any one of claims 1-6 or 24, wherein the allosteric pocket further comprises Arg949, Val952, and Ile1022.

30. The PI3Kα inhibitor of any one of claims 1-6 or 24, wherein the allosteric pocket further comprises Arg949, Tyr1021, and Ile1022.

31. The PI3Kα inhibitor of any one of claims 1-6 or 24, wherein the allosteric pocket further comprises Glu950, Tyr1021, and Ile1022.

32. The PI3Kα inhibitor of any one of claims 1-6 or 24, wherein the allosteric pocket further comprises Val952, Ile1022, and Glu950.

33. The PI3Kα inhibitor of any one of claims 1-6 or 24, wherein the allosteric pocket further comprises Glu950, Tyr1021, and Arg949.

34. The PI3Kα inhibitor of any one of claims 1-6 or 24, wherein the allosteric pocket further comprises Glu950, Ile1022, and Arg949.

35. The PI3Kα inhibitor of any one of claims 1-6, wherein the allosteric pocket further comprises four of Arg949, Glu950, Val952, Tyr1021, and Ile1022.

36. The PI3Kα inhibitor of any one of claims 1-6 or 35, wherein the allosteric pocket further comprises Arg949, Glu950, Val952, and Tyr1021.

37. The PI3Kα inhibitor of any one of claims 1-6 or 35, wherein the allosteric pocket further comprises Glu950, Val952, Tyr1021, and Ile1022.

38. The PI3Kα inhibitor of any one of claims 1-6 or 35, wherein the allosteric pocket further comprises Val952, Tyr1021, Ile1022, and Arg949.

39. The PI3Kα inhibitor of any one of claims 1-6 or 35, wherein the allosteric pocket further comprises Tyr1021, Ile1022, Arg949, and Glu950.

40. The PI3Kα inhibitor of any one of claims 1-6 or 35, wherein the allosteric pocket further comprises Arg949, Glu950, Val952, and Ile1022.

41. The PI3Kα inhibitor of any one of claims 1-6, wherein the allosteric pocket further comprises one or more of Gln809, Leu812, Ile816, Gly912, Leu938, His940, Arg951, Met1010, Glu1012, Leu1013, Asp1018, and Ile1019.

42. The PI3Kα inhibitor of any one of claims 1-6 or 41, wherein the allosteric pocket further comprises one of Gln809, Leu812, Ile816, Gly912, Leu938, His940, Arg951, Met1010, Glu1012, Leu1013, Asp1018, and Ile1019.

43. The PI3Kα inhibitor of any one of claims 1-6 or 42, wherein the allosteric pocket further comprises Gln809.

44. The PI3Kα inhibitor of any one of claims 1-6 or 42, wherein the allosteric pocket further comprises Leu812.

45. The PI3Kα inhibitor of any one of claims 1-6 or 42, wherein the allosteric pocket further comprises Ile816.

46. The PI3Kα inhibitor of any one of claims 1-6 or 42, wherein the allosteric pocket further comprises Gly912.

47. The PI3Kα inhibitor of any one of claims 1-6 or 42, wherein the allosteric pocket further comprises Leu938.

48. The PI3Kα inhibitor of any one of claims 1-6 or 42, wherein the allosteric pocket further comprises His940.

49. The PI3Kα inhibitor of any one of claims 1-6 or 42, wherein the allosteric pocket further comprises Arg951.

50. The PI3Kα inhibitor of any one of claims 1-6 or 42, wherein the allosteric pocket further comprises Met1010.

51. The PI3Kα inhibitor of any one of claims 1-6 or 42, wherein the allosteric pocket further comprises Glu1012.

52. The PI3Kα inhibitor of any one of claims 1-6 or 42, wherein the allosteric pocket further comprises Leu1013.

53. The PI3Kα inhibitor of any one of claims 1-6 or 42, wherein the allosteric pocket further comprises Asp1018.

54. The PI3Kα inhibitor of any one of claims 1-6 or 42, wherein the allosteric pocket further comprises Ile1019.

55. The PI3Kα inhibitor of any one of claims 1-6 or 41, wherein the allosteric pocket further comprises two of Gln809, Leu812, Ile816, Gly912, Leu938, His940, Arg951, Met1010, Glu1012, Leu1013, Asp1018, and Ile1019.

56. The PI3Kα inhibitor of any one of claims 1-6 or 41, wherein the allosteric pocket further comprises three of Gln809, Leu812, Ile816, Gly912, Leu938, His940, Arg951, Met1010, Glu1012, Leu1013, Asp1018, and Ile1019.

57. The PI3Kα inhibitor of any one of claims 1-6 or 41, wherein the allosteric pocket further comprises four of Gln809, Leu812, Ile816, Gly912, Leu938, His940, Arg951, Met1010, Glu1012, Leu1013, Asp1018, and Ile1019.

58. The PI3Kα inhibitor of any one of claims 1-6 or 41, wherein the allosteric pocket further comprises five of Gln809, Leu812, Ile816, Gly912, Leu938, His940, Arg951, Met1010, Glu1012, Leu1013, Asp1018, and Ile1019.

59. The PI3Kα inhibitor of any one of claims 1-6 or 41, wherein the allosteric pocket further comprises six of Gln809, Leu812, Ile816, Gly912, Leu938, His940, Arg951, Met1010, Glu1012, Leu1013, Asp1018, and Ile1019.

60. The PI3Kα inhibitor of any one of claims 1-6 or 41, wherein the allosteric pocket further comprises seven of Gln809, Leu812, Ile816, Gly912, Leu938, His940, Arg951, Met1010, Glu1012, Leu1013, Asp1018, and Ile1019.

61. The PI3Kα inhibitor of any one of claims 1-6 or 41, wherein the allosteric pocket further comprises eight of Gln809, Leu812, Ile816, Gly912, Leu938, His940, Arg951, Met1010, Glu1012, Leu1013, Asp1018, and Ile1019.

62. The PI3Kα inhibitor of any one of claims 1-6 or 41, wherein the allosteric pocket further comprises nine of Gln809, Leu812, Ile816, Gly912, Leu938, His940, Arg951, Met1010, Glu1012, Leu1013, Asp1018, and Ile1019.

63. The PI3Kα inhibitor of any one of claims 1-6 or 41, wherein the allosteric pocket further comprises ten of Gln809, Leu812, Ile816, Gly912, Leu938, His940, Arg951, Met1010, Glu1012, Leu1013, Asp1018, and Ile1019.

64. The PI3Kα inhibitor of any one of claims 1-6 or 41, wherein the allosteric pocket further comprises eleven of Gln809, Leu812, Ile816, Gly912, Leu938, His940, Arg951, Met1010, Glu1012, Leu1013, Asp1018, and Ile1019.

65. The PI3Kα inhibitor of any one of claims 1-6 or 41, wherein the allosteric pocket further comprises Gln809, Leu812, Ile816, Gly912, Leu938, His940, Arg951, Met1010, Glu1012, Leu1013, Asp1018, and Ile1019.

66. A PI3Kα inhibitor compound comprising:
(i) a first, a second, and a third hydrogen bonding moiety each capable of forming hydrogen bonds with Leu911 and/or Lys941 of PI3Kα^{H1047R}, and wherein the first and second, second and third, and first and third hydrogen bonding moieties are each about 2.4 Å apart;
(ii) a hydrophobic moiety about 2.5 Å from the third hydrogen bonding moiety and is capable of interacting with Ile1022 of PI3Kα^{H1047R};
(iii) a first and a second aromatic moiety, wherein the first and second aromatic moieties together comprise a fused bicyclic aromatic ring system or two aromatic rings connected by a single bond, and wherein the first and second aromatic moieties are each capable of forming a pi-pi stacking interaction with Phe937 of PI3Kα^{H1047R};
(iv) a third aromatic moiety capable of forming a cation-pi interaction with Lys941 and/or a pi-pi stacking interaction with Tyr1021 and/or a of PI3Kα^{H1047R}; and
(v) a fourth hydrogen bonding moiety capable of forming hydrogen bonds with Glu950 and/or Arg949;
wherein the PI3Kα inhibitor has a K_{D} for PI3Kα^{H1047R} of about 0.1 nM to about 1 μM.

67. The PI3Kα inhibitor of claim 66, wherein one of a first, a second, or a third hydrogen bonding moiety each capable of forming hydrogen bonds with Leu911 and/or Lys941 of PI3Kα^{H1047R} forms a hydrogen bond with the backbone carbonyl of Gly912.

68. The PI3Kα inhibitor of claim 66 or 67, wherein the first, second, and third hydrogen bonding moieties are each independently selected from —C(═O)—, —CH(OH)—, —C(═NH)—, —CH(NH₂)—, —NH—, —O—, —S(═NH)(O)— and —S(═O)ₘ—; wherein m is 0, 1 or 2; and
wherein the first and second, and second and third hydrogen bonding moieties are not the same.

69. The PI3Kα inhibitor of any one of claims 66-68, wherein the hydrophobic moiety is C1-C6 alkyl or C1-C6 haloalkyl.

70. The PI3Kα inhibitor of any one of claims 66-69, wherein the hydrophobic moiety is C1-C3 alkyl or C1-C3 haloalkyl.

71. The PI3Kα inhibitor of any one of claims 66-70, wherein the hydrophobic moiety is a C1-C3 alkyl.

72. The PI3Kα inhibitor of any one of claims 66-70, wherein the hydrophobic moiety is C1-C3 haloalkyl.

73. A PI3Kα inhibitor compound comprising a first and a second aromatic moiety joined by a linking group to a third aromatic moiety, wherein the first and a second aromatic moieties comprise a fused bicyclic aromatic ring system or two aromatic rings connected by a single bond, wherein:
(i) the linking group is capable of forming hydrogen bonds with Leu911 and/or Lys941 of PI3Kα^{H1047R};
(ii) the first and second aromatic moieties are each capable of forming a pi-pi stacking interaction with Phe937 of PI3Kα^{H1047R}; and
(iii) the third aromatic moiety is capable of forming an optional cation-pi interaction with Lys941 and/or a pi-pi stacking interaction with Tyr1021 of PI3Kα^{H1047R};
wherein the PI3Kα inhibitor has a K_{D} for PI3Kα^{H1047R} of about 0.1 nM to about 1 μM.

74. The PI3Kα inhibitor of claim 73, wherein the linking group is capable of forming water-mediated hydrogen bonds with Leu911 and/or Lys941 of PI3Kα^{H1047R}.

75. The PI3Kα inhibitor of claim 73, wherein the linking group comprises an amide, a urea, an imidazole, a benzimidazole, or a carbamate.

76. The PI3Kα inhibitor of any one of claims 66-75, wherein the first and second aromatic moieties comprise a fused bicyclic aromatic ring system.

77. The PI3Kα inhibitor of claim 76, wherein the fused bicyclic aromatic ring system is a 9-10 membered aromatic ring system.

78. The PI3Kα inhibitor of any one of claims 66-77, wherein the first and second aromatic moieties form a benzimidazolyl or benzofuranyl.

79. The PI3Kα inhibitor of any one of claims 66-78, wherein the third aromatic moiety is a 5-6 membered heteroaryl.

80. The PI3Kα inhibitor of any one of claims 66-79, wherein the third aromatic moiety is a pyrazolyl, oxazolyl, thiazolyl, pyridinyl, or pyrimidinyl.

81. The PI3Kα inhibitor of any one of claims 66-78, wherein the third aromatic moiety is a 9-10 membered heteroaryl.

82. The PI3Kα inhibitor of any one of claims 66-78 and 81, wherein the third aromatic moiety is a 9 membered heteroaryl.

83. The PI3Kα inhibitor of any one of claims 66-78 and 81-82, wherein the third aromatic moiety is selected from benzimidazolyl, purinyl, indazolyl, and imidazopyridinyl.

84. The PI3Kα inhibitor of any one of claims 66-78, wherein the third aromatic moiety is a phenyl.

85. The PI3Kα inhibitor of any one of claims 66-84, wherein the compound is capable of forming a direct binding interaction with an allosteric pocket on PI3Kα.

86. The PI3Kα inhibitor of any one of claims 66-85, wherein the allosteric pocket comprises three or more of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

87. The PI3Kα inhibitor of any one of claims 66-86, wherein the allosteric pocket comprises three of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

88. The PI3Kα inhibitor of any one of claims 66-86, wherein the allosteric pocket comprises four of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

89. The PI3Kα inhibitor of any one of claims 66-86, wherein the allosteric pocket comprises five of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

90. The PI3Kα inhibitor of any one of claims 66-86, wherein the allosteric pocket comprises six of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

91. The PI3Kα inhibitor of any one of claims 66-86, wherein the allosteric pocket comprises seven of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

92. The PI3Kα inhibitor of any one of claims 66-86, wherein the allosteric pocket comprises eight of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

93. The PI3Kα inhibitor of any one of claims 66-86, wherein the allosteric pocket comprises nine of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

94. The PI3Kα inhibitor of any one of claims 66-86, wherein the allosteric pocket comprises ten of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

95. The PI3Kα inhibitor of any one of claims 66-86, wherein the allosteric pocket comprises eleven of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

96. The PI3Kα inhibitor of any one of claims 66-86, wherein the allosteric pocket comprises twelve of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

97. The PI3Kα inhibitor of any one of claims 66-86, wherein the allosteric pocket comprises thirteen of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

98. The PI3Kα inhibitor of any one of claims 66-86, wherein the allosteric pocket comprises fourteen of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

99. The PI3Kα inhibitor of any one of claims 66-86, wherein the allosteric pocket comprises fifteen of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

100. The PI3Kα inhibitor of any one of claims 66-86, wherein the allosteric pocket comprises sixteen of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

101. The PI3Kα inhibitor of any one of claims 66-86, wherein the allosteric pocket comprises seventeen of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

102. The PI3Kα inhibitor of any one of claims 66-86, wherein the allosteric pocket comprises eighteen of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

103. The PI3Kα inhibitor of any one of claims 66-86, wherein the allosteric pocket comprises nineteen of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

104. The PI3Kα inhibitor of any one of claims 66-86, wherein the allosteric pocket comprises twenty of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

105. The PI3Kα inhibitor of any one of claims 66-86, wherein the allosteric pocket comprises twenty one of: Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

106. The PI3Kα inhibitor of any one of claims 66-86, wherein the allosteric pocket comprises Gln809, Leu812, Thr813, Ile816, Leu911, Gly912, Phe937, Leu938, His940, Lys941, Arg949, Glu950, Arg951, Val952, Phe1002, Met1010, Glu1012, Leu1013, Asp1018, Ile1019, Tyr1021, and Ile1022.

107. The PI3Kα inhibitor of any one of claims 1-106, wherein the backbone carbon atoms of Phe937 are displaced by about 4 Å to about 7 Å upon binding to PI3Kα.

108. The PI3Kα inhibitor of any one of claims 1-107, wherein the backbone carbon atoms of Leu911 are displaced by about 5 to about 6 Å upon binding to PI3Kα.

109. The PI3Kα inhibitor of any one of claims 1-108, wherein the compound has an IC₅₀ for PI3Kα^{H1047R} of about 0.1 nM to about 500 nM.

110. The PI3Kα inhibitor of any one of claims 1-109, wherein the compound has an IC₅₀ for PI3Kα^{H1047R} of about 0.1 nM to about 100 nM.

111. The PI3Kα inhibitor of any one of claims 1-110, wherein the compound has an IC₅₀ for PI3Kα^{H1047R} of about 0.1 nM to about 50 nM.

112. The PI3Kα inhibitor of any one of claims 1-111, wherein the compound has an IC₅₀ for PI3Kα^{H1047R} of about 0.1 nM to about 25 nM.

113. The PI3Kα inhibitor of any one of claims 1-112, wherein the compound has an IC₅₀ for PI3Kα^{H1047R} of about 0.1 nM to about 10 nM.

114. The PI3Kα inhibitor of any one of claims 1-113, wherein the compound has a molecule weight of about 275 Da to about 650 Da.

115. The PI3Kα inhibitor of any one of claims 1-114, wherein the compound has a molecule weight of about 300 Da to about 500 Da.

116. The PI3Kα inhibitor of any one of claims 1-115, wherein the compound has a molecule weight of about 350 Da to about 450 Da.

117. The PI3Kα inhibitor of any one of claims 1-116, wherein the compound has a eudysmic ratio of about 8 to about 500.

118. The PI3Kα inhibitor of any one of claims 1-117, wherein the compound has a eudysmic ratio of about 8 to about 75.

119. The PI3Kα inhibitor of any one of claims 1-117, wherein the compound has a eudysmic ratio of about 50 to about 200.

120. The PI3Kα inhibitor of any one of claims 1-117, wherein the compound has a eudysmic ratio of about 150 to about 300.

121. The PI3Kα inhibitor of any one of claims 1-117, wherein the compound has a eudysmic ratio of about 250 to about 500.

122. The PI3Kα inhibitor of any one of claims 1-121, wherein the compound has a K_{D} for PI3Kα^{H1047R} of about 0.1 nM to about 50 nM.

123. The PI3Kα inhibitor of any one of claims 1-122, wherein the compound has a K_{D} for PI3Kα^{H1047R} of about 0.1 nM to about 25 nM.

124. The PI3Kα inhibitor of any one of claims 1-123, wherein the compound has a K_{D} for PI3Kα^{H1047R} of about 0.1 nM to about 10 nM.

125. The PI3Kα inhibitor of any one of claims 1-124, wherein the compound is about 1-fold to about 50-fold selective for PI3Kα^{H1047R} over wild type PI3Kα.

126. The PI3Kα inhibitor of any one of claims 1-125, wherein the compound is about 3-fold to about 10-fold selective for PI3Kα^{H1047R} over wild type PI3Kα.

127. The PI3Kα inhibitor of any one of claims 1-126, wherein the compound is about 10-fold to about 50-fold selective for PI3Kα^{H1047R} over wild type PI3Kα.

128. The PI3Kα inhibitor of any one of claims 1-127, wherein the compound has the structure of Formula (I-A): 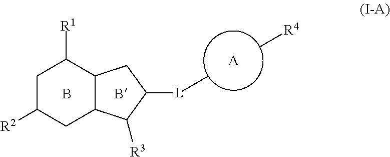
wherein:
Ring A is a 5-10 membered ring system substituted at a distal position with R⁴, wherein the ring system is selected from cycloalkyl, heterocyclyl, aryl, and heteroaryl;
Rings B and B′ together form a 9 membered ring system selected from cycloalkyl, heterocyclyl, aryl, and heteroaryl;
R¹ and R² are independently hydrogen, a hydrogen bond acceptor, or a hydrophobic moiety;
R³ is a hydrophobic moiety;
R⁴ comprises a hydrogen bond donor and/or a hydrogen bond acceptor; and
L comprises two or three groups selected from —C(═O)—, —CH(OH)—, —C(═NH)—, —CH(NH₂)—, —NH—, —O—, and —S(O)ₙ—; wherein n is 0, 1, or 2; and
wherein no two adjacent groups in the L moeity are the same.

129. The PI3Kα inhibitor of any one of claims 1-128, wherein the compound of Formula (I) has the structure of Formula (I-B): 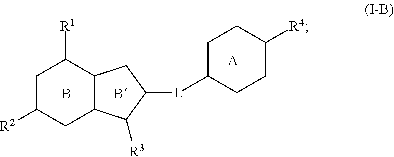
wherein Ring A is a phenyl, cyclohexyl, 6 membered heterocyclyl, or 6 membered heteroaryl.

130. The PI3Kα inhibitor of claim 128 or 129, wherein L forms one or more hydrogen bonds with Leu911 and/or Lys941 of PI3Kα^{H1047R}.

131. The PI3Kα inhibitor of any one of claims 1-128, wherein the compound of Formula (I) has the structure of Formula (I-C): 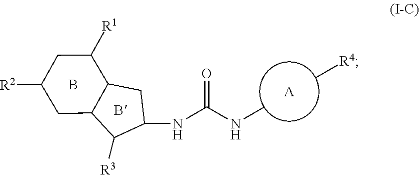
wherein Ring A is a 5-10 membered ring selected from aryl, cycloalkyl, heterocyclyl, and heteroaryl.

132. The PI3Kα inhibitor of any one of claims 1-128, wherein the compound of Formula (I) has the structure of Formula (I-D): 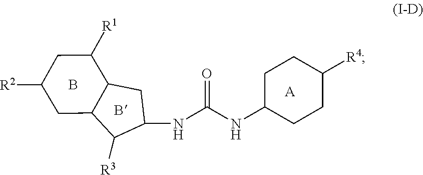
wherein Ring A is a phenyl, cyclohexyl, 6 membered heterocyclyl, or 6 membered heteroaryl.

133. The PI3Kα inhibitor of any one of claims 1-131, wherein the compound has a Log P value from about 1 to about 6.

134. The PI3Kα inhibitor of any one of claims 1-132, wherein the compound has a Log P value from about 1 to about 5.

135. The PI3Kα inhibitor of any one of claims 1-133, wherein the compound has a Log P value from about 1 to about 3.

136. The PI3Kα inhibitor of any one of claims 1-134, wherein the compound has a Log P value from about 1 to about 2.

137. The PI3Kα inhibitor of any one of claims 128-136, wherein Ring B and/or Ring B′ form a pi-pi stacking interaction with Phe937 of PI3Kα^{H1047R}.

138. The PI3Kα inhibitor of any one of claims 128-137, wherein Ring A is an aryl or heteroaryl ring which forms a cation-pi interaction with Lys941.

139. The PI3Kα inhibitor of any one of claims 128-137, wherein Ring A is an aryl or heteroaryl ring which forms a pi-pi stacking interaction with Tyr1021 of PI3Kα^{H1047R}.

140. The PI3Kα inhibitor of any one of claims 128-139, wherein the PI3Kα inhibitor has a K_{D} for PI3Kα^{H1047R} of about 0.1 nM to about 1 μM.

141. The PI3Kα inhibitor of any one of claims 128-139, wherein the PI3Kα inhibitor has a K_{D} for PI3Kα^{H1047R} of about 0.1 nM to about 500 nM.

142. The PI3Kα inhibitor of any one of claims 128-139, wherein the PI3Kα inhibitor has a K_{D} for PI3Kα^{H1047R} of about 0.1 nM to about 100 nM.

143. A method of identifying a PI3Kα inhibitor compound comprising:
(i) screening in silico a library for candidate compounds capable of forming a direct binding interaction with an allosteric pocket on PI3Kα, wherein a three-dimensional model of the binding site on PI3Kα is computationally derived from the atomic coordinates in Table 1; and
(ii) evaluating the candidate compounds identified in step (i) in one or more in vitro or in vivo assays for their ability to bind to the PI3Kα allosteric pocket to thereby identify the PI3Kα inhibitor;
wherein the PI3Kα inhibitor compound has a K_{D} for PI3Kα^{H1047R} of about 0.1 nM to about 1 μM.

144. A method of identifying a PI3Kα inhibitor compound comprising:
(i) using the atomic coordinates in Table 2 to generate a 3-dimensional model of PI3Kα;
(ii) identifying three or more residues of an allosteric binding pocket;
(ii) generating a specific 3-dimensional target using the three or more allosteric binding pocket residues;
(iii) employing the specific 3-dimensional target to select a potential inhibitor of PI3Kα;
(iv) obtaining the potential inhibitor of PI3Kα; and
(v) contacting the potential inhibitor of PI3Kα with PI3Kα^{H1047R} in vitro to determine a K_{D} for PI3Kα^{H1047R}, wherein if the K_{D} for PI3Kα^{H1047R} is about 0.1 nM to about 1 μM, the potential inhibitor of PI3Kα is a PI3Kα inhibitor.

145. The method of claim 143 or 144, wherein the PI3Kα inhibitor compound is a compound of any one of claims 1-142.

146. A method of inhibiting PI3Kα activity, comprising identifying a compound capable of forming a direct binding interaction with an allosteric pocket on PI3Kα;
wherein if the K_{D} for PI3Kα^{H1047R} is about 0.1 nM to about 1 μM, the compound is a PI3Kα inhibitor;
wherein the allosteric pocket corresponds to the atomic coordinates in Table 1; and
contacting a PI3Kα^{H1047R} protein with the PI3Kα inhibitor compound.

147. The method of claim 146, wherein the compound is a compound of any one of claims 1-142.

148. A pharmaceutical composition comprising the compound of any one of claims 1-142, and one or more pharmaceutically acceptable excipients.

149. A method of treating cancer in a subject in need thereof comprising administering to the subject a therapeutically effective amount of the compound of any one of claims 1-142, or the pharmaceutical composition of claim 148.

150. The method of claim 149, wherein the cancer is a PI3Kα-associated cancer.

151. The method of claim 149 or 150, wherein the cancer is a PI3Kαα^{H1047R}-associated cancer.
